SEQUENCE LISTING (21 sequences)

SEQ ID NO: 1 - protein, 3936 aa, Homo sapiens
MTTMLQKSDSNASFLRAARAGNLDKVVEYLKGGIDINTCNQNGLNALHLAAKEGHVGLVQELLGRGSSVDSATKKGNTALHIASLAGQAEVVKVLVKEGANINAQSQNGFTPLYMAAQENHIDVVKYLLENGANQSTATEDGFTPLAVALQQGHNQAVAILLENDTKGKVRLPALHIAARKDDTKSAALLLQNDHNADVQSKMMVNRTTESGFTPLHIAAHYGNVNVATLLLNRGAAVDFTARNGITPLHVASKRGNTNMVKLLLDRGGQIDAKTRDGLTPLHCAARSGHDQVVELLLERGAPLLARTKNGLSPLHMAAQGDHVECVKHLLQHKAPVDDVTLDYLTALHVAAHCGHYRVTKLLLDKRANPNARALNGFTPLHIACKKNRIKVMELLVKYGASIQAITESGLTPIHVAAFMGHLNIVLLLLQNGASPDVTNIRGETALHMAARAGQVEVVRCLLRNGALVDARAREEQTPLHIASRLGKTEIVQLLLQHMAHPDAATTNGYTPLHISAREGQVDVASVLLEAGAAHSLATKKGFTPLHVAAKYGSLDVAKLLLQRRAAADSAGKNGLTPLHVAAHYDNQKVALLLLEKGASPHATAKNGYTPLHIAAKKNQMQIASTLLNYGAETNIVTKQGVTPLHLASQEGHTDMVTLLLDKGANIHMSTKSGLTSLHLAAQEDKVNVADILTKHGADQDAHTKLGYTPLIVACHYGNVKMVNFLLKQGANVNAKTKNGYTPLHQAAQQGHTHIINVLLQHGAKPNATTANGNTALAIAKRLGYISVVDTLKVVTEEVTTTTTTITEKHKLNVPETMTEVLDVSDEEGDDTMTGDGGEYLRPEDLKELGDDSLPSSQFLDGMNYLRYSLEGGRSDSLRSFSSDRSHTLSHASYLRDSAVMDDSVVIPSHQVSTLAKEAERNSYRLSWGTENLDNVALSSSPIHSGFLVSFMVDARGGAMRGCRHNGLRIIIPPRKCTAPTRVTCRLVKRHRLATMPPMVEGEGLASRLIEVGPSGAQFLGKLHLPTAPPPLNEGESLVSRILQLGPPGTKFLGPVIVEIPHFAALRGKERELVVLRSENGDSWKEHFCDYTEDELNEILNGMDEVLDSPEDLEKKRICRIITRDFPQYFAVVSRIKQDSNLIGPEGGVLSSTVVPQVQAVFPEGALTKRIRVGLQAQPMHSELVKKILGNKATFSPIVTLEPRRRKFHKPITMTIPVPKASSDVMLNGFGGDAPTLRLLCSITGGTTPAQWEDITGTTPLTFVNECVSFTTNVSARFWLIDCRQIQESVTFASQVYREIICVPYMAKFVVFAKSHDPIEARLRCFCMTDDKVDKTLEQQENFAEVARSRDVEVLEGKPIYVDCFGNLVPLTKSGQHHIFSFFAFKENRLPLFVKVRDTTQEPCGRLSFMKEPKSTRGLVHQAICNLNITLPIYTKESESDQEQEEEIDMTSEKNDETESTETSVLKSHLVNEVPVLASPDLLSEVSEMKQDLIKMTAILTTDVSDKAGSIKVKELVKAAEEEPGEPFEIVERVKEDLEKVNEILRSGTCTRDESSVQSSRSERGLVEEEWVIVSDEEIEEARQKAPLEITEYPCVEVRIDKEIKGKVEKDSTGLVNYLTDDLNTCVPLPKEQLQTVQDKAGKKCEALAVGRSSEKEGKDIPPDETQSTQKQHKPSLGIKKPVRRKLKEKQKQKEEGLQASAEKAELKKGSSEESLGEDPGLAPEPLPTVKATSPLIEETPIGSIKDKVKALQKRVEDEQKGRSKLPIRVKGKEDVPKKTTHRPHPAASPSLKSERHAPGSPSPKTERHSTLSSSAKTERHPPVSPSSKTEKHSPVSPSAKTERHSPASSSSKTEKHSPVSPSTKTERHSPVSSTKTERHPPVSPSGKTDKRPPVSPSGRTEKHPPVSPGRTEKRLPVSPSGRTDKHQPVSTAGKTEKHLPVSPSGKTEKQPPVSPTSKTERIEETMSVRELMKAFQSGQDPSKHKTGLFEHKSAKQKQPQEKGKVRVEKEKGPILTQREAQKTENQTIKRGQRLPVTGTAESKRGVRVSSIGVKKEDAAGGKEKVLSHKIPEPVQSVPEEESHRESEVPKEKMADEQGDMDLQISPDRKTSTDFSEVIKQELEDNDKYQQFRLSEETEKAQLHLDQVLTSPFNTTFPLDYMKDEFLPALSLQSGALDGSSESLKNEGVAGSPCGSLMEGTPQISSEESYKHEGLAETPETSPESLSFSPKKSEEQTGETKESTKTETTTEIRSEKEHPTTKDITGGSEERGATVTEDSETSTESFQKEATLGSPKDTSPKRQDDCTGSCSVALAKETPTGLTEEAACDEGQRTFGSSAHKTQTDSEVQESTATSDETKALPLPEASVKTDTGTESKPQGVIRSPQGLELALPSRDSEVLSAVADDSLAVSHKDSLEASPVLEDNSSHKTPDSLEPSPLKESPCRDSLESSPVEPKMKAGIFPSHFPLPAAVAKTELLTEVASVRSRLLRDPDGSAEDDSLEQTSLMESSGKSPLSPDTPSSEEVSYEVTPKTTDVSTPKPAVIHECAEEDDSENGEKKRFTPEEEMFKMVTKIKMFDELEQEAKQKRDYKKEPKQEESSSSSDPDADCSVDVDEPKHTGSGEDESGVPVLVTSESRKVSSSSESEPELAQLKKGADSGLLPEPVIRVQPPSPLPSSMDSNSSPEEVQFQPVVSKQYTFKMNEDTQEEPGKSEEEKDSESHLAEDRHAVSTEAEDRSYDKLNRDTDQPKICDGHGCEAMSPSSSAAPVSSGLQSPTGDDVDEQPVIYKESLALQGTHEKDTEGEELDVSRAESPQADCPSESFSSSSSLPHCLVSEGKELDEDISATSSIQKTEVTKTDETFENLPKDCPSQDSSITTQTDRFSMDVPVSDLAENDEIYDPQITSPYENVPSQSFFSSEESKTQTDANHTTSFHSSEVYSVTITSPVEDVVVASSSSGTVLSKESNFEGQDIKMESQQESTLWEMQSDSVSSSFEPTMSATTTVVGEQISKVIITKTDVDSDSWSEIREDDEAFEARVKEEEQKIFGLMVDRQSQGTTPDTTPARTPTEEGTPTSEQNPFLFQEGKLFEMTRSGAIDMTKRSYADESFHFFQIGQESREETLSEDVKEGATGADPLPLETSAESLALSESKETVDDEADLLPDDVSEEVEEIPASDAQLNSQMGISASTETPTKEAVSVGTKDLPTVQTGDIPPLSGVKQISCPDSSEPAVQVQLDFSTLTRSVYSDRGDDSPDSSPEEQKSVIEIPTAPMENVPFTESKSKIPVRTMPTSTPAPPSAEYESSVSEDFLSSVDEENKADEAKPKSKLPVKVPLQRVEQQLSDLDTSVQKTVAPQGQDMASIAPDNRSKSESDASSLDSKTKCPVKTRSYTETETESRERAEELELESEEGATRPKILTSRLPVKSRSTTSSCRGGTSPTKESKEHFFDLYRNSIEFFEEISDEASKLVDRLTQSEREQEIVSDDESSSALEVSVIENLPPVETEHSVPEDIFDTRPIWDESIETLIERIPDENGHDHAEDPQDEQERIEERLAYIADHLGFSWTELARELDFTEEQIHQIRIENPNSLQDQSHALLKYWLERDGKHATDTNLVECLTKINRMDIVHLMETNTEPLQERISHSYAEIEQTITLDHSEGFSVLQEELCTAQHKQKEEQAVSKESETCDHPPIVSEEDISVGYSTFQDGVPKTEGDSSATALFPQTHKEQVQQDFSGKMQDLPEESSLEYQQEYFVTTPGTETSETQKAMIVPSSPSKTPEEVSTPAEEEKLYLQTPTSSERGGSPIIQEPEEPSEHREESSPRKTSLVIVESADNQPETCERLDEDAAFEKGDDMPEIPPETVTEEEYIDEHGHTVVKKVTRKIIRRYVSSEGTEKEEIMVQGMPQEPVNIEEGDGYSKVIKRVVLKSDTEQSEDNNE

SEQ ID NO: 2 - DNA, 19 bp, Artificial
  siRNA sequence
ggccagaagatctcaagga

SEQ ID NO: 3 - DNA, 19 bp, Artificial
  siRNA sequence
gctgtgtagcattttaaca

SEQ ID NO: 4 - DNA, 19 bp, Artificial
  siRNA sequence
ggcccgaagagctcaagga

SEQ ID NO: 5 - DNA, 62 bp, Mus musculus
  misc_feature (18)..(18): n is g or a
agcccagcagcagcactntctctcctcaaatgcaccaggagccagttcaacaagatttctca

SEQ ID NO: 6 - DNA, 60 bp, Mus musculus
  misc_feature (10)..(10): n is c or t
atcattaggnggtacgtttcctctgatggcacagagaaggaggaggttaccatgcaggga

SEQ ID NO: 7 - DNA, 201 bp, Homo sapiens
  misc_feature (101)..(101): n is a or g
gttgaaagggtatttatttactctttccttttcttctaaatactctctactcttccttctctcttttttccatcttgcatggcatcttggggcggaaaggnatcagagtcagatcaagaacaggaggaagaggtaattttatgacagtgtcacttgttatcggctgtgtcattgctgtaaccactaataagagcacatagt

SEQ ID NO: 8 - DNA, 201 bp, Homo sapiens
  misc_feature (101)..(101): n is c or t
ctttctttgaatgaatcagtactgtggttcctctcctgtcatagacaacctttggccattctgtttttgaccttctccagatccacaggatgagcaggaanggatcgaggaaaggctggcttatattgctgatcaccttggcttcagctggacaggtaaaaagaatgtgacccaggttttcaacaaaacctgacatagatg

SEQ ID NO: 9 - DNA, 201 bp, Homo sapiens
  misc_feature (101)..(101): n is t or a
tatcaaaaatttagtaaggcagttgagtgaaagagatttttaagagtacctctcagacataataaatgctgtttctctaatgtgtcagataccaacctcgntgaatgtctcaccaagatcaaccgaatggatattgttcatctcatggagaccaacacagaacctctccaggagcgcatcagtcatagttatgcagaaatt

SEQ ID NO: 10 - DNA, 201 bp, Homo sapiens
  misc_feature (101)..(101): n is a or c
ctcaccaagatcaaccgaatggatattgttcatctcatggagaccaacacagaacctctccaggagcgcatcagtcatagttatgcagaaattgaacagancattacactggatcatagtgaaggtcaaactgtgtgtgtgtatgtgtgtgtgtgtgtgtgtgtgtgtgtgtgttgtgtctgtgtgtggttaattgaggca

SEQ ID NO: 11 - DNA, 51 bp, Homo sapiens
  misc_feature (27)..(27): n is c or a
ctgagggggacagctcagcaacagcantctttccccaaactcacaaggagc

SEQ ID NO: 12 - DNA, 201 bp, Homo sapiens
  misc_feature (101)..(101): n is c or a
atcgtctcagaggaagacatttctgttggttattccacttttcaggatggcgtccccaaaactgagggggacagctcagcaacagcactctttccccaaantcacaaggagcaagttcaacaggatttctcagggaaaatgcaagacctgcctgaagagtcatctctggaatatcagcaggaatatttgtgagtttccaaa

SEQ ID NO: 13 - DNA, 201 bp, Homo sapiens
  misc_feature (101)..(101): n is c or t
acaaacttcctgtttaaaatttatcaattccatggtactgtcacacaaaaataagatacacaaatgaaatacatttcaggttactaggaaaatcattaggnggtatgtatcctctgaaggcacagagaaagaagagattatggtgcagggaatgccacaggaacctgtcaacatcgaggaaggggatggctattccaaagt

SEQ ID NO: 14 - DNA, 201 bp, Homo sapiens
  misc_feature (101)..(101): n is t or c
ctgtttaaaatttatcaattccatggtactgtcacacaaaaataagatacacaaatgaaatacatttcaggttactaggaaaatcattaggcggtatgtancctctgaaggcacagagaaagaagagattatggtgcagggaatgccacaggaacctgtcaacatcgaggaaggggatggctattccaaagttataaagcg

SEQ ID NO: 15 - DNA, 167 bp, Homo sapiens
  misc_feature (104)..(104): n is g or a
atttcaggttactaggaaaatcattaggcggtatgtatcctctgaaggcacagagaaagaagagattatggtgcagggaatgccacaggaacctgtcaacatcnaggaaggggatggctattccaaagttataaagcgtgttgtattgaagagtgacaccgagcagt

SEQ ID NO: 16 - DNA, 201 bp, Homo sapiens
  misc_feature (101)..(101): n is g or a
atatcaaaaatttagtaaggcagttgagtgaaagagatttttaagagtacctctcagacataataaatgctgtttctctaatgtgtcagataccaacctcnttgaatgtctcaccaagatcaaccgaatggatattgttcatctcatggagaccaacacagaacctctccaggagcgcatcagtcatagttatgcagaaat

SEQ ID NO: 17 - DNA, 201 bp, Homo sapiens
  misc_feature (101)..(101): n is g or a
ttgaatgaatcagtactgtggttcctctcctgtcatagacaacctttggccattctgtttttgaccttctccagatccacaggatgagcaggaacggatcnaggaaaggctggcttatattgctgatcaccttggcttcagctggacaggtaaaaagaatgtgacccaggttttcaacaaaacctgacatagatgcatcag

SEQ ID NO: 18 - DNA, 201 bp, Homo sapiens
  misc_feature (101)..(101): n is g or c
tacctccagaccccaacatccagcgagcggggaggctctcccatcatacaagaacccgaagagccctcagagcacagagaggagagctctccgcggaaaancagcctcgtaatagtggagtctgccgataaccagcctgagacctgtgaaagactcgatgaagatgcagcttttgaaaaggtaagacattcctctccactt

SEQ ID NO: 19 - DNA, 201 bp, Homo sapiens; identical to SEQ ID NO: 18
  misc_feature (101)..(101): n is c or a

SEQ ID NO: 20 - DNA, 201 bp, Homo sapiens
  misc_feature (101)..(101): n is g or a
caatataggtaagcttcaactaaatacttaaatcattctgcctttagggttctcggtacttcaagaggagttatgcactgcacagcacaagcagaaagagnagcaagctgtttctaaagaaagtgagacctgcgatcaccctcctatcgtctcagaggaagacatttctgttggttattccacttttcaggatggcgtccc

SEQ ID NO: 21 - DNA, 201 bp, Homo sapiens
  misc_feature (101)..(101): n is a or c
cagataggggtgatgattctcccgattcttccccagaagaacagaaatcagtaatcgagattcctactgcacccatggagaatgtgccttttactgaaagnaaatccaaaattcctgtaaggactatgcccacttccaccccagcacctccatctgcagagtatgagagttcagtttctgaagattttctatccagtgtag

(19) United States
(12) Patent Application Publication
(10) Pub. No.: US 2013/0059778 A1
(43) Pub. Date: Mar. 7, 2013

(54) Identification of a Genetic Risk Factor for Diabetes

(71) Applicants: Vann Bennett, Hillsborough, NC (US); Jane Healy, Boston, MA (US)

(72) Inventors: Vann Bennett, Hillsborough, NC (US); Jane Healy, Boston, MA (US)

(21) Appl. No.: 13/634,935

(22) PCT Filed: Mar. 15, 2011
(86) PCT No.: PCT/US2011/028458

§ 371 Date: Nov. 7, 2012

Related U.S. Application Data
(60) Provisional application No. 61/313,976, filed on Mar. 15, 2010.

(51) Int. Cl. C12Q 1/68; C12Q 1/54; G06F 17/30; A61K 38/26; A61K 38/17; A61P 3/10; G01N 33/566; C40B 30/04

(52) U.S. Cl. 514/7.2; 435/6.11; 435/7.92; 435/14; 506/9; 514/6.9; 707/769; 707/E17.014

(57) ABSTRACT

Loss of function ankyrin-B variants have impaired function in pancreatic islets and are associated with type 2 diabetes. This finding provides the basis for methods of identifying at-risk individuals for type 2 diabetes and for personalized therapeutic strategies.

### FIELD OF THE INVENTION

The present invention relates to methods of identifying subjects at risk for developing type 2 diabetes as well as methods of treating for subjects at risk for developing or that have type 2 diabetes.

### BACKGROUND OF THE INVENTION

Postprandial insulin secretion reflects the aggregate influence of glucose stimulation of pancreatic beta cells and regulation by neurotransmitters, neuropeptides, and enteric hormones. Amongst the regulators of insulin secretion, vagal release of acetylcholine is involved in normal glucose tolerance, as it augments glucose-stimulated insulin secretion during the passage of food through the gastrointestinal tract (1). Though the wide ranging effects of parasympathetic agonists and the large number of muscarinic receptor isoforms have historically made parasympathetic effects on islet function difficult to interpret, recent work using a beta cell-specific muscarinic receptor-3 (M3) knockout mouse demonstrates both impaired glucose tolerance and reduced insulin secretion in response to both glucose and the muscarinic agonist carbachol (3). Acetylcholine stimulates muscarinic receptors, thereby initiating a cascade of second messenger signaling that results in the activation of Gq-dependent release of inositol-trisphosphate (InsP₃), the stimulation of inositol-trisphosphate receptors (InsP₃R), the release of Ca²⁺ from endoplasmic reticulum (ER) stores, and the exocytosis of insulin-containing granules (2-4). InsP₃Rs bind to ankyrin-B, and in mouse cardiomyocytes, the disruption of ankyrin-B-mediated InsP3R localization and stabilization is accompanied by elevated Ca²⁺ transients (5, 6). Human ankyrin-B mutations that disrupt InsP3 receptor stabilization in cardiomyocytes result in a cardiac arrhythmia syndrome that includes sinus node dysfunction and catecholamine-induced sudden cardiac death (7, 8).

Diabetes mellitus is a chronic disease affecting approximately ten percent of the United States population over the age of 20 and is rapidly increasing in prevalence. Diabetes falls into two general categories: Type I diabetes, a relatively rare autoimmune disease, where blood glucose is abnormal due to lack of insulin, and type 2 diabetes, comprising 95 percent of the cases, where blood glucose is abnormal either due to insulin resistance and/or a defect in insulin secretion. The rising prevalence of type 2 diabetes is alarming given its physical and monetary consequences. Diabetes is a leading cause of blindness, limb loss, peripheral neuropathy, and renal failure in the United States. Diabetes is also associated with a reduced lifespan and an increased risk for cardiovascular disease. In 2002, the Centers for Disease Control and Prevention estimated that the total annual cost of diabetes to the United States health care system was 132 billion dollars.

The impact of adult onset type 2 diabetes on the United States population underscores the importance of identification of genetic risk factors. Thus, there is a long-felt need in the art for methods for identifying subjects at risk for developing type 2 diabetes, which will facilitate intervention to prevent the development of type 2 diabetes and provide targeted strategies for treating such individuals after the onset of disease.

### SUMMARY OF THE INVENTION

The present invention is based, in part, on the discovery that ankyrin-B functions in pancreatic beta cells where it stabilizes the InsP₃R (inositol triphosphate receptor) and is involved in normal calcium release and enhanced insulin secretion in response to muscarinic agonists. Ankyrin-B-haploinsufficient mice exhibit hyperglycemia after oral ingestion but not after intraperitoneal injection of glucose, consistent with impaired parasympathetic potentiation of glucose-stimulated insulin secretion. Further, loss of function ankyrin-B variants have impaired function in pancreatic islets and are associated with type 2 diabetes. This finding provides a method of identifying at-risk individuals and for personalized therapeutic strategies.

Accordingly, as one aspect, the invention provides a method of identifying a subject as having an increased risk of developing type 2 diabetes. In representative embodiments, the method comprises detecting in the subject the presence or absence of an ankB loss of function allele, wherein the presence of an ankB loss of function allele identifies the subject as having an increased risk of developing type 2 diabetes.

In further representative embodiments, the method comprises: correlating the presence or absence of an ankB loss of function allele with the risk of developing type 2 diabetes; and determining the presence or absence of the ankB loss of function allele in the subject, wherein the presence of the ankB loss of function allele identifies the subject as having an increased risk of developing type 2 diabetes.

As still another aspect, the invention provides a method of treating a subject with type 2 diabetes. In representative embodiments, the method comprises: identifying a subject with type 2 diabetes and an ankB loss of function allele; and administering an agent that enhances a glucagon-like peptide 1 (glp-1) signaling pathway to the subject, thereby treating a subject with type 2 diabetes. Optionally, the method further comprises detecting the presence of an ankB loss of function allele in the subject with type 2 diabetes.

As yet another aspect, the invention provides a method of correlating an ankB loss of function allele with the risk of developing type 2 diabetes in a subject. In representative embodiments, the method comprises: detecting the presence of the ankB loss of function allele in a plurality of subjects with type 2 diabetes to determine the prevalence of the ankB loss of function allele in the plurality of diabetic subjects; and correlating the prevalence of the ankB loss of function allele with development of type 2 diabetes, thereby correlating the ankB loss of function allele with the risk of developing type 2 diabetes in a subject.

Also provided is a method of correlating the presence of an ankB loss of function allele with an effective treatment for preventing the development of type 2 diabetes in a subject that has the ankB loss of function allele. In representative embodiments, the method comprises: administering a treatment to a subject (or plurality of subjects) having the ankB loss of function allele; and correlating the presence of the ankB loss of function allele with the effectiveness of the treatment for preventing the development of type 2 diabetes in the subject (or plurality of subjects).

Further encompassed by the invention is a method of correlating the presence of an ankB loss of function allele with an effective treatment for type 2 diabetes in a subject that has the ankB loss of function allele. In representative embodiments, the method comprises: administering a treatment to a subject (or plurality of subjects) with type 2 diabetes and the ankB loss of function allele; determining the effectiveness of the treatment for treating type 2 diabetes in the subject (or plurality of subjects); and correlating the presence of the ankB loss of function allele with the effectiveness of the treatment for type 2 diabetes.

A still further aspect of the invention is a computer-assisted method of identifying an effective treatment for type 2 diabetes in a subject having an ankB loss of function allele that is associated with type 2 diabetes. In representative embodiments, the method comprises: (a) storing a database of biological data for a plurality of subjects, the biological data that is being stored including for each of said plurality of subjects: (i) a treatment type, (ii) an ankB loss of function allele associated with type 2 diabetes, and (iii) at least one clinical measure for type 2 diabetes from which treatment efficacy can be determined; and then (b) querying the database to determine the effectiveness of a treatment type in treating type 2 diabetes in a subject having an ankB loss of function allele, thereby identifying an effective treatment for type 2 diabetes in a subject having an ankB loss of function allele associated with type 2 diabetes.

As another aspect, the invention provides a method of correlating an ankB loss of function allele with a good or poor prognosis for a subject having type 2 diabetes. In representative embodiments, the method comprises: detecting the presence or absence of the ankB loss of function allele in a subject (or plurality of subjects) with type 2 diabetes; and correlating the presence or absence of the ankB loss of function allele with a good or poor prognosis for type 2 diabetes in the subject (or plurality of subjects), thereby correlating the ankB loss of function allele with a good or poor prognosis for type 2 diabetes in a subject (or plurality of subjects).

Yet another aspect of the invention is a method of identifying a subject with type 2 diabetes as having a good or a poor disease prognosis. In representative embodiments, the method comprises: correlating the presence or absence of an ankB loss of function allele with a good or a poor prognosis for type 2 diabetes in a subject (or plurality of subjects); and determining the presence or absence of the ankB loss of function allele in a subject (or plurality of subjects), wherein the presence or absence of the ankB loss of function allele identifies the subject (or plurality of subjects) as having a good or a poor disease prognosis.

Unless the context indicates otherwise, it is specifically intended that the various features of the invention described herein can be used in any combination.

Moreover, the present invention also contemplates that in some embodiments of the invention, any feature or combination of features set forth herein can be excluded or omitted.

These and other aspects of the invention are addressed in more detail in the description of the invention set forth below.

### BRIEF DESCRIPTION OF THE DRAWINGS

FIG. 1. Amino acid sequence of human ankyrin-B (NCBI database Accession No. GI:119626696).

FIG. 2A-F. Ankyrin-B co-localizes with InsP3R and is required for its stability. A. Pancreas from a C57-B6 mouse co-stained with anti-ANK B and InsP3R antibodies. B. Pancreases from neonatal ankB (+/+), (+/−), (−/−) mice co-stained with anti-InsP3R and islet-marker insulin. C. Quantification of mean intra-islet InsP3R staining (n=6, *p=0.05, **p=0.01). Levels are a percentage of wild type staining. D. Immunoblot of INS-1 823/3 cell lysates treated with ank B-specific siRNA (ankB siRNA 1 and 2), control (ctl) siRNA, or no siRNA (untreated). Blots show expression of ankyrin-B (ANK B), InsP3R, K-ATP channel subunit (KIR6.2), dihydropyridine receptor (DHPR) or loading control GAPDH. E. Protein turnover of InsP3R and GAPDH as a measure of expression change 0, 2, 4, 6, and 8 hours after cycloheximide (Cx) treatment. Graph of mean protein expression from ankyrin-B (ankB) or ctl siRNA treated INS-1 cells were quantified and graphed (**p=0.01, n=4). Protein expression is given as a percentage of untreated levels. InsP3R and GAPDH turnover in ankB siRNA treated cells (red and gray lines), and ctl siRNA treated cells (pink and black lines). F. Representative immunoblot of turnover experiment.

FIGS. 3A-F. Characteristics of ankyrin-B expression in B6 mouse islets and ankyrin-B and InsP3R expression in ankB mouse islets. A. Ankyrin-B is enriched in beta cells of the endocrine pancreas. Top two panels show co-localization of ankyrin-B and ankyrin-G with beta cell marker insulin in sections of B6 mouse pancreas. Bottom two panels show localization of ankyrin-B and somatostatin (SS) and glucagon, markers of alpha and delta cells, respectively. B. Neonatal ankB (+/+), (+/−), (−/−) mouse pancreas sections co-stained with ankyrin-B and insulin antibodies. C. Representative immunoblot of ankyrin-B (ANK B) and GAPDH expression in adult ankB (+/+) and (+/−) mouse islet lysates. D. Quantification of islet ankyrin-B expression in adult ankB mice (n=3). E. Pancreases from adult ankB (+/+) and (+/−) mice co-stained with anti-InsP3R and insulin. F. Quantification of mean intra-islet InsP3R staining (n=3, *p=0.05). Levels are a percentage of wild type staining.

FIGS. 4A-G. Ankyrin-B deficiency reduces carbachol stimulated insulin secretion and intraislet calcium release. A. Insulin secretion assay using islets from ankB(+/−) and (+/+) mice or B. rat islets treated with ankB or ctl siRNA containing adenovirus. Graphs depict secretion response to basal or stimulatory glucose (3.3 or 16.7 mM glu) or 16.7 mM glucose plus 0.1 mM carbachol (Cch)(n=6). C. Insulin secretion assay using rat islets treated with adenovirus expressing siRNA-resistant human ankyrin B (h ankB), ankB siRNA, and/or ctl siRNA. Presence (−/−) or absence (−) of each virus is indicated. Insulin secretion is represented as fold response relative to 8 mM glucose (n=6). Intraislet calcium levels in Fura-2 loaded ankB(+/−) (red) and ankB(+/+) (black) islets. Responses to 0.1 μM Cch in buffer containing 0 mM calcium/EGTA (Cch, D) or 5 mM calcium (Cch+CaCl₂, E), potassium chloride (KCl, F), or stimulatory glucose (16.7 mM Glu, G) are shown. The top panels are representative experiments depicting calcium response as 355/380 ratio over time. Bottom panels show mean peak-baseline values+/−SEM for each stimulus. Data represent recordings from 3-7 islets/animal for 6 animals/genotype (*p=0.05,**p=0.01, n.s.=not significant).

FIGS. 5A-D. Effect of ankyrin B knockdown on expression of InsP3 receptor and muscarinic receptor (M3R). A. Quantitative PCR of InsP3R gene subtypes 1-3 (ITPR1-3) in INS-1 832/3 cells. B. Quantitative PCR of the predominant subtypes of ITPR 1 and 3 in 823/3 cells treated with ankB siRNA (ankB siRNA 1 and 2), ctl siRNA, or no siRNA (untreated) (n=3). C. Representative immunoblot of ankyrin-B knockdown in rat islet lysates. M3R expression and GAPDH control expression are also shown. D. Representative immunoblot of ankyrin-B expression levels in lysates of islets treated with adenoviruses expressing GFP, ctl or ankB siRNA, full length FLAG-tagged human ankyrin-B or ankyrinB RAN (h ankB or h ankB RAN). Presence (+) or absence (−) of each virus is indicated.

FIGS. 6A-E. Metabolic characteristics and islet parameters for ankyrin-B (+/−) mice. A. Effect of ankyrin-B deficiency on dynamic insulin release in response to stimulatory glucose and carbachol. Islets isolated from ankB (+/−) mice (n=3) and the wild-type control mice (n=3) were subject to perifusion and insulin release was performed. Average % of basal insulin values±SEM is shown every 2 minutes. Bars above the traces indicate the duration of stimulation. Effects of stimulation of 0.1 mM Carbachol (CCh) were shown during 20-minute perifusion with 11 mM glucose. Top right panel shows the area under the curve (AUC) for the 1^{st}-phase insulin release during the 1^{st} 10 minutes after stimulation with 11 mM glucose. Middle right panel shows AUC for the 2^{nd}-phase insulin release during the secondary 10 minutes after stimulation with 11 mM glucose. Bottom right panel shows the AUC for insulin release after stimulation with 0.1 mM CCh and 11 mM glucose. Average values±SEM are shown. Arbitrary unit is shown for AUC. * indicates P value less than 0.05. B. Fasting glucose levels and C. body weights of 16 to 20-month-old ankB mice. Data represent the mean for 10 animals/genotype. D. Islet morphometric analysis of islets, including size and density as determined by immunofluorescence quantification of pancreas sections treated with insulin antibody, and total pancreatic insulin content, measured by insulin RIA of acid ethanol extracted pancreas. Data represent the mean+/−SEM for 6 animals/genotype. E. Representative examples of islets stained with insulin antibody used in the morphometric analysis.

FIGS. 7A-H. AnkB(+/−) mice demonstrate postprandial hypoinsulinemic hyperglycemia. A. Intraperitoneal glucose tolerance test (IP GTT): blood glucose levels following i.p. administration of glucose (2 mg/g). Oral glucose tolerance test B. (ORAL GTT): blood glucose levels following oral administration of glucose (2 mg/g). C. Quantified area under the curve (AUC) for oral GTT. D. Mean serum insulin levels (ng/mL) in mice before (fasted) and 30 min after (fed) glucose administration (IP or oral). E. Insulin tolerance test (ITT): blood glucose levels measured following the i.p. administration of insulin (0.75 U/kg). Data are expressed as a percentage of initial glucose level. F-H. Glp-1 measurements: F. Insulin secretion assay using islets from ankB(+/−) and (+/+) mice or G. rat islets treated with ankB or ctl siRNA containing adenovirus. Insulin secretion is represented as Glp-1 fold response relative to 16.7 mM glucose (n=6). H. Mean serum Glp-1 levels (ng/mL) in mice before (fasted) and 30 min after (fed) oral glucose administration. All measurements in A-E and H were performed on 16-22 week old littermates (n=6 per genotype), **p=0.01,***p=0.001, n.s.=not significant.

FIGS. 8A-E. R1788W ankyrin-B is enriched in diabetics and fails to rescue carbachol-stimulated insulin secretion. A. Case-control study of severe ankyrin-B mutations in a GENNID sample population. Top panel shows GENNID probands screened. Racial diabetes prevalence is given as an absolute value and a percentage of total. The bottom panel shows the point mutation tested (AA change), the corresponding genomic nucleotide change (SNP), and the number of heterozygotes identified. B. Partial pedigrees of the R/W heterozygotes identified in the association study (Circle=female; square=male; black=diabetic; white=nondiabetic; *=R/W heterozygote). C. Clustal-W protein sequence alignment of ankyrin-B shows conservation of R-1788. D. Insulin secretion assay using rat islets treated with adenovirus expressing GFP, ankB or ctl siRNA, siRNA-resistant human ankyrin B (h ankB), and/or human ankyrin B containing the R/W mutation (h ankB R/W). Insulin secretion is represented as fold response relative to 8 mM glucose. (n=6, **p=0.03, n.s.=not significant). E. Competition assay measuring ability of wild type or R/W ankyrin-B (h ankB or h ankB R/W) to displace ¹²⁵I labelled-InsP3R from immobilized GST-conjugated ankyrin-B membrane-binding domain (ankB MBD). Coomassie gel shows protein input. Scatchard analysis shows ankyrin-B-InsP3R interactions (¹²⁵I InsP₃R tetramer bound as a percentage of control).

### DETAILED DESCRIPTION OF THE INVENTION

The present invention will now be described with reference to the accompanying drawings, in which representative embodiments of the invention are shown. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention. All publications, patent applications, patents, and other references mentioned herein are incorporated by reference in their entirety.

### Definitions

The following terms are used in the description herein and the appended claims:

The singular forms “a,” “an” and “the” are intended to include the plural forms as well, unless the context clearly indicates otherwise.

Furthermore, the term “about,” as used herein when referring to a measurable value such as an amount or the length of a polynucleotide or polypeptide sequence, dose, time, temperature, and the like, is meant to encompass variations of ±20%, ±10%, ±5%, ±1%, ±0.5%, or even ±0.1% of the specified amount.

Also as used herein, “and/or” refers to and encompasses any and all possible combinations of one or more of the associated listed items, as well as the lack of combinations when interpreted in the alternative (“or”).

Unless the context indicates otherwise, it is specifically intended that the various features of the invention described herein can be used in any combination.

Moreover, the present invention also contemplates that in some embodiments of the invention, any feature or combination of features set forth herein can be excluded or omitted.

To illustrate, if the specification states that a complex comprises components A, B and C, it is specifically intended that any of A, B or C, or a combination thereof, can be omitted and disclaimed.

As used herein, the transitional phrase “consisting essentially of” is to be interpreted as encompassing the recited materials or steps “and those that do not materially affect the basic and novel characteristic(s)” of the claimed invention (e.g., DNA demethylase activity). See, In re Herz, 537 F.2d 549, 551-52, 190 U.S.P.Q. 461, 463 (CCPA 1976) (emphasis in the original); see also MPEP §2111.03. Thus, the term “consisting essentially of” as used herein should not be interpreted as equivalent to “comprising.”

As used herein, the terms “reduce,” “reduces,” “reduced,” “reducing,” “reduction” as well as “inhibit,” “inhibits,” “inhibiting,” inhibition,” “inhibitor” and similar terms indicate a decrease in the specified parameter, e.g., of at least about 25%, 35%, 50%, 75%, 80%, 85%, 90%, 95%, 97% or more. In particular embodiments, the reduction results in no or essentially no (i.e., an insignificant amount, for example, less than about 10% or even 5%) detectable activity.

As used herein, the terms “enhance,” “enhances,” “enhancing,” “enhancer,” “enhancement” as well as “increase,” “increases,” “increasing” and similar terms indicate an elevation in the specified parameter, e.g., of at least about 25%, 50%, 75%, 100%, 150%, 200%, 300%, 400%, 500% or more.

As used herein, “increased risk” (and similar terms) refers to an enhanced level of risk that a subject has of developing type 2 diabetes as compared with a suitable control subject (e.g., matched for age, gender, race, ethnicity, body mass and the like), for example, a control subject that does not have the ankB loss of function allele or a control subject that does not have any ankB loss of function allele.

A “sample” can be any biological sample containing nucleic acid and/or protein of a subject. Nonlimiting examples of a sample according to the present invention include a cell, a body fluid (blood or plasma, semen, urine), a tissue (e.g., skin), a washing, a swabbing (e.g., a mouth swab), etc. as would be well known in the art.

As used herein, “nucleic acid” encompasses both RNA and DNA, including cDNA, genomic DNA, synthetic (e.g., chemically synthesized) DNA and chimeras of RNA and DNA. The nucleic acid may be double-stranded or single-stranded. Where single-stranded, the nucleic acid may be a sense strand or an antisense strand. The nucleic acid may be synthesized using oligonucleotide analogs or derivatives (e.g., inosine or phosphorothioate nucleotides). Such oligonucleotides can be used, for example, to prepare nucleic acids that have altered base-pairing abilities or increased resistance to nucleases.

An “allele” as used herein is one of a series of different forms (i.e., variants) of a gene. In other words, alleles are alternative DNA sequences at the same physical locus on the chromosome. A population or species of organisms typically includes multiple alleles at each locus among various individuals. In any particular diploid organism, with two copies of each chromosome, the genotype for each gene is determined by the pair of alleles present at that locus. If the alleles are the same, the organism is homozygous at that locus; if the alleles are different, the organism is heterozygous. As known in the art, certain alleles may have a higher or lower frequency, or even be absent, in particular ethnic, racial and/or geographic populations.

An ankB “loss of function” allele is an allele that encodes an ankyrin-B protein having at least one function reduced (or even undetectable) as compared with the predominant ankyrin-B in the population (e.g., the human ankyrin-B with the amino acid sequence provided by NCBI database Accession No. GI:119626696). The ankyrin-B function that is reduced can be any ankyrin-B function, including but not limited to localization of InsP₃R in pancreatic beta cells, localization of InsP₃R in cardiomyocytes, parasympathetic augmentation (e.g., with a muscarinic agonist such as carbachol) of glucose-stimulated insulin secretion by pancreatic beta cells, parasympathetic augmentation (e.g., with a muscarinic agonist such as carbachol) of intracellular calcium release (e.g., in pancreatic beta cells), stabilization of InsP₃R (e.g., in pancreatic beta cells), interaction of ankyrin-B with co-chaperone hsp40 (e.g., in pancreatic beta cells), or any combination of the foregoing (i.e., the ankB loss of function “phenotype”).

Subjects according to the present invention include both avians and mammals. Mammalian subjects include but are not limited to humans, non-human mammals, non-human primates (e.g., monkeys, chimpanzees, baboons, etc.), dogs, cats, mice, hamsters, rats, guinea pigs, horses, cows, pigs, rabbits, sheep and goats. Avian subjects include but are not limited to chickens, turkeys, ducks, geese, quail and pheasant, and birds kept as pets (e.g., parakeets, parrots, macaws, cockatoos, and the like). In particular embodiments, the subject is a laboratory animal (e.g., an animal model of type 2 diabetes). Human subjects include neonates, infants, juveniles, adults (for example, subjects of about 18, 20, 25, 30, 40, 45, 50 or 55 years of age of older) and/or geriatric subjects (for example, subjects of about 60, 65, 70 or 75 years of age and older). In some embodiments of the invention, the subject has type 2 diabetes. In some embodiments of the invention, the subject does not have type 2 diabetes. In some embodiments of the invention, the subject has a family history of type 2 diabetes (e.g., in first-degree genetically related family members or first-, second- and/or third-degree genetically related family members). In some embodiments of the invention, subjects include males and/or females.

With respect to human subjects, in representative embodiments, the subject is Caucasian (e.g., white, European and/or of European ancestry), African and/or of African ancestry (e.g., black, African-American), Asian (including, for example, Chinese, Japanese, Indian, Korean and/or Middle Eastern [e.g., Israeli] populations and the like) and/or of Asian ancestry, Pacific Islander and/or of Pacific Islander ancestry, American Indian and/or of American Indian ancestry, Hispanic (e.g., Mexican, Argentine and/or Brazilian populations and the like) and/or Hispanic ancestry, and the like. In some embodiments of the invention, subjects include subjects that are heterozygous and/or homozygous for an ankB loss of function allele.

Subjects at risk for type 2 diabetes or that have type 2 diabetes encompass human subjects at risk for or who have type 2 diabetes as well as animal subjects at risk for or that exhibit one or more of the clinical, physiological and/or biochemical indicia of type 2 diabetes (e.g., an animal model of type 2 diabetes) such as insulin resistance, hyperglycemia, and the like as is well known in the art.

By the terms “treat,” “treating” or “treatment of” (and grammatical variations thereof) it is meant that the severity of the subject's condition is reduced, at least partially improved or stabilized and/or that some alleviation, mitigation, decrease or stabilization in at least one clinical symptom and/or parameter is achieved and/or there is a delay in the progression of the disease or disorder.

The terms “prevent,” “preventing” and “prevention” (and grammatical variations thereof) refer to avoidance, prevention and/or delay of the onset of a disease, disorder and/or a clinical symptom(s) in a subject and/or a reduction in the severity of the onset of the disease, disorder and/or clinical symptom(s) relative to what would occur in the absence of the methods of the invention. The prevention can be complete, e.g., the total absence of the disease, disorder and/or clinical symptom(s). The prevention can also be partial, such that the occurrence of the disease, disorder and/or clinical symptom(s) in the subject and/or the severity of onset is less than what would occur in the absence of the methods of the present invention.

An “effective amount,” as used herein, refers to an amount that imparts a desired effect, which is optionally a therapeutic or prophylactic effect.

A “treatment effective” amount as used herein is an amount that is sufficient to provide some improvement or benefit to the subject. Alternatively stated, a “treatment effective” amount is an amount that will provide some alleviation, mitigation, decrease or stabilization in at least one clinical symptom in the subject. Those skilled in the art will appreciate that the therapeutic effects need not be complete or curative, as long as some benefit is provided to the subject.

A “prevention effective” amount as used herein is an amount that is sufficient to prevent and/or delay the onset of a disease, disorder and/or clinical symptoms in a subject and/or to reduce and/or delay the severity of the onset of a disease, disorder and/or clinical symptoms in a subject relative to what would occur in the absence of the methods of the invention. Those skilled in the art will appreciate that the level of prevention need not be complete, as long as some benefit is provided to the subject.

A “diagnostic method”, as used herein, refers to a screening procedure that is carried out to identify those subjects that are affected or likely to be affected with a particular disorder. A “diagnostic method” need not be definitive or conclusive in identifying a subject and may be carried out in conjunction with, preceded and/or followed up by additional diagnostic measures.

A “prognostic method” refers to a method used to predict, at least in part, the course and/or severity of the disease. For example, a prognostic method may be carried out to both identify an affected individual, to evaluate the severity of the disease, and/or to predict the future course of the disease. Such methods may be useful in evaluating the necessity for therapeutic treatment, what type of treatment to implement, and the like. In addition, a prognostic method may be carried out on a subject previously diagnosed with a particular disorder when it is desired to gain greater insight into how the disease will progress for that particular subject and/or the likelihood that a particular patient will respond favorably to a particular drug treatment, or when it is desired to classify or separate patients into distinct and different sub-populations for the purpose of treatment and/or conducting a clinical trial. A “prognostic method” need not be definitive or conclusive and may be carried out in conjunction with, preceded and/or or followed up by additional prognostic measures.

Use of ankB Loss of Function Alleles as a Risk Factor for Type II Diabetes.

As one aspect, the invention provides a method of identifying a subject with reduced parasympathetic augmentation (e.g., with a muscarinic agonist such as carbachol) of glucose stimulated insulin secretion by pancreatic beta cells as compared with a suitable control subject (e.g., matched for age, gender, ethnicity, race and/or body mass and the like), for example, a control subject that does not have the ankB loss of function allele carried by the subject or a control subject that does not have any ankB loss of function allele. In particular embodiments, the invention comprises detecting in a subject the presence or absence of an ankB loss of function allele, wherein the presence of an ankB loss of function allele identifies the subject as having reduced parasympathetic augmentation of glucose stimulated insulin secretion by pancreatic beta cells as compared with a suitable control subject (as defined in the preceding sentence).

The present invention also provides methods of identifying a subject as having an increased risk of developing type 2 diabetes. In particular embodiments, the method comprises detecting in a subject the presence or absence of an ankB loss of function allele, wherein the presence of an ankB loss of function allele identifies the subject as having an increased risk of developing type 2 diabetes. In representative embodiments of the invention, the method comprises detecting in a subject the presence or absence of an ankB loss of function allele, wherein the absence of an ankB loss of function allele indicates that the subject does not have an increased risk of developing type 2 diabetes due to the presence of an ankB loss of function allele.

In other exemplary embodiments, the invention provides a method of identifying a subject as having an increased risk of developing type 2 diabetes. As a non-limiting illustration, in representative embodiments, the method comprises: correlating the presence or absence of an ankB loss of function allele with the risk of developing type 2 diabetes; and determining the presence or absence of the ankB loss of function allele in the subject, wherein the presence of the ankB loss of function allele identifies the subject as having an increased risk of developing type 2 diabetes. In embodiments of the invention, the method comprises: correlating the presence or absence of an ankB loss of function allele with the risk of developing type 2 diabetes; and determining the presence or absence of the ankB loss of function allele in the subject, wherein the absence of the ankB loss of function allele in the subject identifies the subject as not having an increased risk of developing type 2 diabetes due to the presence of the ankB loss of function allele.

In embodiments, the presence of an ankB loss of function allele further identifies the subject as suitable for a particular treatment regimen to reduce the risk of type 2 diabetes developing in the subject, for example, a treatment that reduces postprandial glycemic levels. Accordingly, as one option, the method can further comprise placing the subject identified as at risk for developing type 2 diabetes on a treatment that reduces postprandial glycemic levels. Methods of reducing postprandial glycemic levels are known in the art and include dietary modifications (e.g., a low glycemic diet, optionally including a high fiber content) and/or exercise.

In particular embodiments of the invention, the presence of an ankB loss of function allele further identifies the subject as suitable for treatment with an agent that enhances a glucagon-like peptide 1 (glp1) signaling pathway. Optionally, the method further comprises administering an agent that enhances a glp-1 signaling pathway to the subject identified as at risk for developing type 2 diabetes. Agents that enhance a glp1 signaling pathway are known in the art and include glp1 agonists and analogs (e.g., Exenatide and Exendin-4 [marketed as Byetta® by Eli Lilly], Liraglutide [Novo Nordisk], Albiglutide [GlaxoSmithKline] and Taspoglatide [Roche]) as well as dipeptidyl dipeptidase-IV (DPP-IV) inhibitors (e.g., vildagliptin [Novartis], sitagliptin [marketed as Januvia® by Merck], saxagliptin [Bristol-Myers Squibb, AstraZeneca], linagliptin [Boehringer-Ingelheim], Alogliptin [Takeda], and berberine [herbal supplement with DPP-IV inhibitor included).

In some embodiments, the method further comprises administering a gastric inhibitory peptide (GIP) analog to the subject identified as at risk for developing type 2 diabetes, GIP analogs are known in the art and include, for example, GIP analogs as described in U.S. Pat. No. 6,921,748; an amino-terminal modified Tyr¹ glucitol GIP (O'Harte et al., (1999) Diabetes 48:758-765), and N-9-fluroenylmethoxycarbonyl-GIP and N-palmitate-GIP (Gault et al., (2002) Biochem J. 367(Pt 3):913-920).

The invention also contemplates methods of treating a subject with type 2 diabetes. In particular embodiments, the method comprises identifying a subject with type 2 diabetes and an ankB loss of function allele (e.g., an ankB loss of function allele that is associated with an increased risk of developing type 2 diabetes), and administering an agent that enhances the glp-1 signaling pathway and/or a GIP analog to the subject and/or administering a treatment that reduces postprandial glycemic levels), thereby treating a subject with type 2 diabetes. In representative embodiments, the method further comprises detecting the presence of an ankB loss of function allele(s) in the subject with type 2 diabetes. As an alternative, the subject may already be identified as having an ankB loss of function allele(s).

The invention also encompasses a method of correlating an ankB loss of function allele with a good or poor prognosis for a subject having type 2 diabetes. In exemplary embodiments, the method comprises: detecting the presence or absence of the ankB loss of function allele in a plurality of subjects with type 2 diabetes; and correlating the presence or absence of the ankB loss of function allele with a good or poor prognosis for type 2 diabetes in the plurality of subjects, thereby correlating the ankB loss of function allele with a good or poor prognosis for type 2 diabetes in a subject.

The invention further provides methods of determining the prognosis for a subject with type 2 diabetes, e.g., a method of identifying a subject with type 2 diabetes as having a good or a poor disease prognosis. In exemplary embodiments, the method comprises detecting the presence or absence in a subject with type 2 diabetes of an ankB loss of function allele, wherein the presence of an ankB loss of function allele identifies the subject as having a good or a poor disease prognosis

In other embodiments, the invention provides a method of determining the prognosis of a subject with type 2 diabetes, the method comprising: correlating the presence or absence of an ankB loss of function allele with a good or a poor prognosis for type 2 diabetes; and determining the presence or absence of the ankB loss of function allele in a subject, wherein the presence or absence of the ankB loss of function allele identifies the subject as having a good or a poor disease prognosis.

Methods of assessing disease outcome for subjects with type 2 diabetes to determine prognosis are known in the art and may be based on any of a number of clinical indicia known by those of ordinary skill in the art (e.g., insulin resistance, hyperglycemia, hyperinsulinemia and/or vascular complications including cardiovascular disease, ocular disease and renal disease).

The invention further encompasses methods of correlating an ankB loss of function allele with the risk of developing type 2 diabetes. One approach to making such a correlation is based on population studies. Such population based studies can be retrospective and/or prospective. For example, in some embodiments, the invention provides a method of correlating an ankB loss of function allele with the risk of developing type 2 diabetes in a subject, the method comprising: detecting the presence of the ankB loss of function allele in a plurality of subjects with type 2 diabetes to determine the prevalence of the ankB loss of function allele in the plurality of diabetic subjects; and correlating the prevalence of the ankB loss of function allele with development of type 2 diabetes, thereby correlating the ankB loss of function allele with the risk of developing type 2 diabetes in a subject. In exemplary embodiments, heterozygosity and/or homozygosity for the ankB loss of function allele is correlated with the risk of developing type 2 diabetes. Optionally, the method can further comprise comparing the prevalence of the ankB loss of function allele in the plurality of subjects with type 2 diabetes with the prevalence of the ankB loss of function allele in a reference population (e.g., a plurality of subjects that do not have type 2 diabetes or a plurality of subjects from a general population). As a further option, standard statistical techniques known to those skilled in the art can be employed to determine if there is a statistically significant difference in the prevalence of the ankB loss of function allele in the subject population with type 2 diabetes as compared with the prevalence in a reference population. Those skilled in the art will appreciate that the reference population can comprise matched subjects, e.g., for gender, age, ethnicity and/or race.

In other embodiments, a prospective approach is used. For example, in some embodiments, the invention provides a method of correlating an ankB loss of function allele with the risk of developing type 2 diabetes in a subject, the method comprising: detecting the presence or absence of the ankB loss of function allele in a plurality of subjects that do not have type 2 diabetes; following the plurality of subjects over time; determining the incidence of type 2 diabetes in the subjects that have the ankB loss of function allele (heterozygous and/or homozygous), and optionally the incidence of type 2 diabetes in the subjects that do not have the ankB loss of function allele; and correlating the incidence of type 2 diabetes in the plurality of subjects with the presence or absence of the ankB loss of function allele, thereby correlating the ankB loss of function allele with the risk of developing type 2 diabetes in a subject. In exemplary embodiments, heterozygosity and/or homozygosity for the ankB loss of function allele is correlated with the risk of developing type 2 diabetes. Optionally, the method can further comprise comparing the incidence of type 2 diabetes in the subjects with an ankB loss of function allele with the incidence of type 2 diabetes in a reference population (e.g., a plurality of subjects that do not have an ankB loss of function allele or a plurality of subjects from a general population). As a further option, standard statistical techniques known to those skilled in the art can be employed to determine if there is a statistically significant difference in the incidence of type 2 diabetes in the subjects with an ankB loss of function allele as compared with the incidence in a reference population. Those skilled in the art will appreciate that the reference population can comprise matched subjects, e.g., for gender, age, ethnicity and/or race.

Pedigree analysis can also be used to determine a correlation between an ankB loss of function allele and risk of developing type 2 diabetes using standard methods known to those skilled in the art. Pedigree analysis can also be used to strengthen or confirm a correlation identified using other techniques such as population-based studies as described in the preceding paragraph and as are well-known in the art. For example, the method can comprise identifying a family with two or more cases of type 2 diabetes and/or other disorders associated with an ankB loss of function allele (e.g., cardiac arrhythmia such as type 4 long QT syndrome also known as sick sinus syndrome with bradycardia), for example, two or more cases in first, second and/or third degree genetically-related family members, determining the inheritance of the ankB loss of function allele in some or all of the family members, and correlating the presence of one (heterozygous) and/or two (homozygous) copies of the ankB loss of function allele in a subject with the development of type 2 diabetes.

In representative embodiments, once a correlation between an ankB loss of function allele and type 2 diabetes has been determined, the method can further comprise: detecting the presence or absence of the ankB loss of function allele in a subject (e.g., a subject that does not have type 2 diabetes or has not been diagnosed with type 2 diabetes); and determining whether or not the subject has an increased risk of developing type 2 diabetes. For example, if pedigree analysis determines that an ankB loss of function allele is correlated with the incidence of type 2 diabetes in a family, then other individuals within the family can be tested for the presence or absence of the ankB loss of function allele (heterozygous and/or homozygous) to determine whether or not they are at an increased risk for developing type 2 diabetes. As another illustration, if population-based studies determine that an ankB loss of function allele is associated with the risk of developing type 2 diabetes in a population of subjects, other individuals (e.g., similarly situated to the test population and/or in another population) can be tested for the presence or absence of the ankB loss of function allele (heterozygous and/or homozygous) to determine whether or not they are at an increased risk of developing type 2 diabetes.

The invention also provides methods of correlating the presence of an ankB loss of function allele with an effective treatment for preventing the development of type 2 diabetes or for treating type 2 diabetes in a subject that has the ankB loss of function allele (e.g., “personalized medicine” to identify treatments more likely to be effective in preventing and/or treating diabetes in a subject that has an ankB loss of function allele). In some embodiments, the invention provides a method of correlating the presence of an ankB loss of function allele with an effective treatment for preventing the development of type 2 diabetes in a subject that has an ankB loss of function allele, the method comprising: administering a treatment to a subject that has an ankB loss of function allele; and correlating the presence of the ankB loss of function allele with the effectiveness of the treatment for preventing the development of type 2 diabetes. The invention may be advantageously carried out in a population of subjects having an ankB loss of function allele (e.g., the same ankB loss of function allele) by correlating the presence of an ankB loss of function allele with the effectiveness of a treatment for preventing the development of type 2 diabetes in the population of subjects (or a subpopulation thereof). Those skilled in the art will appreciate that when assessing a population as a whole, a correlation may be found for the entire population (or subpopulations thereof), although there may be no benefit for particular individuals within the population. In representative embodiments, the method further comprises determining the effectiveness of the treatment. In addition, the method can optionally comprise comparing the effectiveness of the treatment in a subject or (sub)population of subjects having an ankB loss of function allele(s) with the effectiveness in a reference population (e.g., subjects that have the ankB loss of function allele or subjects that have any ankB loss of function allele, where the subject is not administered the treatment, for example, the subject is not provided with any treatment or is provided with a different treatment regimen). It will further be appreciated by the skilled worker that this embodiment of the invention can be carried out prospectively and/or retrospectively using data acquired from a previously treated subject or (sub)population of subjects.

The invention also provides a method of correlating the presence of an ankB loss of function allele with an effective treatment for type 2 diabetes in a subject that has an ankB loss of function allele. In representative embodiments, the method comprises: administering a treatment to the subject with type 2 diabetes and an ankB loss of function allele; determining the effectiveness of the treatment for treating type 2 diabetes in the subject; and correlating the presence of the ankB loss of function allele with the effectiveness of the treatment for type 2 diabetes. This aspect of the invention may be advantageously carried out in a population of subjects having an ankB loss of function allele (e.g., the same ankB loss of function allele) by correlating the presence of an ankB loss of function allele with an effective treatment for type 2 diabetes in the population of subjects (or a subpopulation thereof). Those skilled in the art will appreciate that when assessing a population as a whole, a correlation may be found for the entire population (or subpopulations thereof), although there may be no benefit for particular individuals within the population.

In representative embodiments, the method further comprises determining the effectiveness of the treatment. In addition, the method can optionally comprise comparing the effectiveness of the treatment in a subject or (sub)population of subjects having an ankB loss of function allele(s) with the effectiveness in a reference population (e.g., subjects with type 2 diabetes that do not have the ankB loss of function allele or subjects with type 2 diabetes and the ankB loss of function allele or subjects that have type 2 diabetes and any ankB loss of function allele, where the subjects are not administered the treatment, for example, they are not provided with any treatment or are provided with a different treatment regimen). It will further be appreciated by the skilled worker that this embodiment of the invention can be carried out prospectively and/or retrospectively using data acquired from a previously treated subject or (sub)population of subjects.

Treatment regimens for type 2 diabetes are well-known in the art and include without limitation, administration of insulin, administration of an oral hypoglycemic agent, dietary modification and/or exercise.

Subjects that respond well to a particular treatment protocol can be analyzed for the presence or absence of one or more ankB loss of function alleles and a correlation can be established according to methods known in the art and as described herein. Likewise, subjects that respond poorly to a particular treatment protocol can also be analyzed for the presence or absence of one or more ankB loss of function alleles and a correlation can be established between the one or more ankB loss of function alleles and the poor response. Then, a subject that is a candidate for prevention or treatment of type 2 diabetes can be assessed for the presence or absence of the appropriate ankB loss of function allele associated with a good and/or poor response to a particular treatment(s), and an appropriate treatment regimen can be determined, and optionally provided.

In some embodiments, the methods of correlating an ankB loss of function allele with the effectiveness of a treatment regimen can be carried out using a computer database. Thus, the invention further comprises a computer-assisted method of identifying an effective treatment for type 2 diabetes in a subject having an ankB loss of function allele that is associated with type 2 diabetes. In embodiments of the invention, the method comprises: (a) storing a database of biological data for a plurality of subjects, the biological data that is being stored including for each of said plurality of subjects: (i) a treatment type, (ii) an ankB loss of function allele(s) associated with type 2 diabetes, and (iii) at least one clinical measure for type 2 diabetes from which treatment efficacy can be determined; and then (b) querying the database to determine the effectiveness of a treatment in treating type 2 diabetes in a subject having an ankB loss of function allele(s), thereby identifying an effective treatment for type 2 diabetes in a subject having an ankB loss of function allele associated with type 2 diabetes.

A correlation can be established using any suitable method. In general, identifying a correlation involves an analysis that establishes a statistical association (e.g., a statistically significant association) between the presence or absence of one or more ankB loss of function alleles and the relevant parameter(s). An analysis that identifies a statistical association (e.g., a statistically significant association) between the presence or absence of one or more ankB loss of function alleles and the specified parameter(s) establishes a correlation between the presence or absence of the one or more ankB loss of function alleles and the particular parameter being evaluated.

An ankB loss of function allele includes any such allele now known or later identified. A number of ankB loss of function alleles are already known in the art. In particular embodiments, the loss of function allele encodes an ankyrin-B precursor and/or mature polypeptide comprising a substitution, insertion (including duplications) and/or deletion (including truncations) of one or more amino acids as compared with the predominant functional ankyrin-B protein (e.g., NCBI database Accession No. GI:119626696). In representative embodiments, the loss of function allele encodes an ankyrin-B protein comprising a substitution of about 1, 2, 3, 4, 5, 6, 8, 10, 12, 20, 30, 50 or more amino acids, an insertion of about 1, 2, 3, 4, 5, 6, 7, 8, 10, 12, 15, 20, 30, 50 or more amino acids and/or a deletion of about 1, 2, 3, 4, 5, 6, 7, 8, 9, 10, 12, 20, 30, 50 or more amino acids. In embodiments of the invention, the allele comprises a nonsense mutation (i.e., a pre-mature stop codon), a missense mutation (i.e., a change in one or more amino acids) and/or a frame-shift mutation. The substitution, insertion and/or deletion can optionally be in the membrane binding, spectrin binding, death and/or carboxy terminal domains of the ankyrin-B protein (see, e.g., U.S. Pat. No. 7,144,706).

The modification resulting in the ankyrin-B loss of function phenotype can be in the ankB coding sequence (i.e., exons), intron regions, upstream non-coding sequences (e.g., promoter and/or enhancer elements) and/or downstream non-coding sequences that result in a loss of function phenotype. Modifications that are not in protein coding regions can still result in impairments in transcription, translation and/or gene splicing, and the like such that the allele expresses less or even no detectable ankryin-B precursor and/or mature polypeptide. In embodiments of the invention, an ankB loss of function allele in a non-coding region comprises a substitution of about 1, 2, 3, 4, 5, 6, 8, 10, 12, 20, 30, 50 or more nucleotides, an insertion of about 1, 2, 3, 4, 5, 6, 7, 8, 10, 12, 15, 20, 30, 50 or more nucleotides and/or a deletion of about 1, 2, 3, 4, 5, 6, 7, 8, 9, 10, 12, 20, 30, 50 or more nucleotides as compared with a wild-type or the predominant ankB allele (e.g., the human sequence at ENSG00000145362 in the ensemble database).

In representative embodiments, the ankB loss of function allele comprises one, two, three, four or more SNPs that result in the loss of function phenotype, and which will optionally result in a change in amino acid sequence (i.e., a non-synonymous SNP).

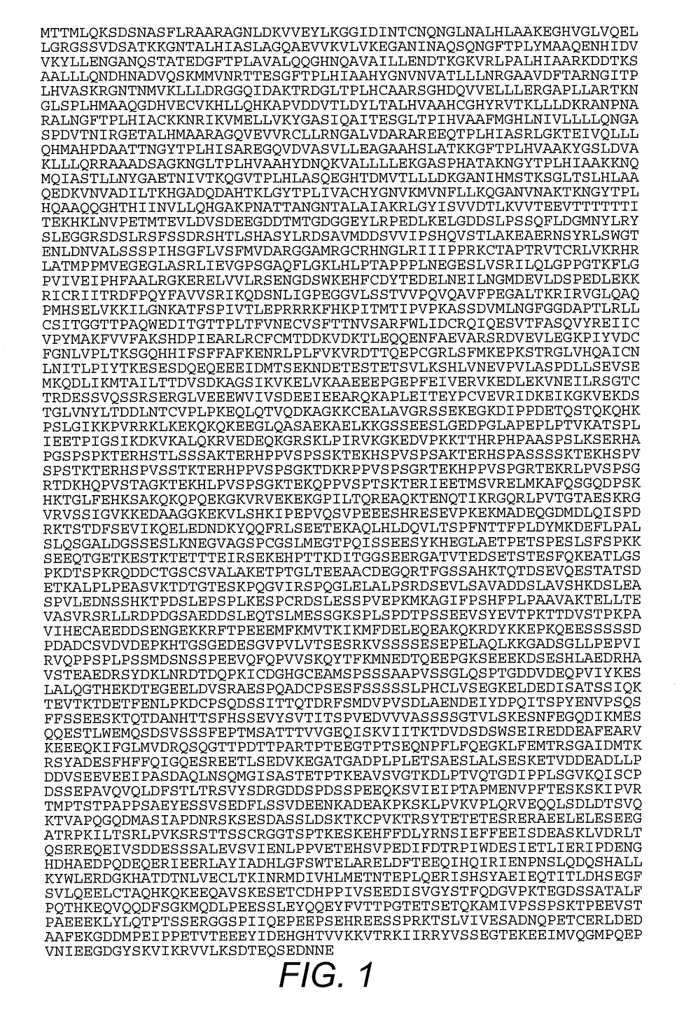

In embodiments of the invention, the ankB loss of function allele is a human ankB loss of function allele and encodes/results in an ankryin-B polypeptide comprising one or more substitutions, insertions and/or deletions (each as described in the preceding paragraph) as compared with the amino acid sequence of NCBI database Accession No. GI:119626696 (FIG. 1; SEQ ID NO:1).

To illustrate, in exemplary embodiments a human ankB loss of function allele results in (the encoded protein comprises):
(a) a glutamic acid to glycine substitution at amino acid position 1425 of ankyrin-B relative to NCBI database Accession No. GI:119626696;
(b) an arginine to tryptophan substitution at amino acid position 1450 of ankyrin-B relative to NCBI database Accession No. GI:119626696;
(c) a valine to aspartic acid substitution at amino acid position 1516 of ankyrin-B relative to NCBI database Accession No. GI:119626696;
(d) a threonine to asparagine substitution at amino acid position 1552 of ankyrin-B relative to NCBI database Accession No. GI:119626696;
(e) a leucine to isoleucine substitution at amino acid position 1622 of ankyrin-B relative to NCBI database Accession No. GI:119626696;
(f) a threonine to asparagine substitution at amino acid position 1626 of ankyrin-B relative to NCBI database Accession No. GI:119626696;
(g) an arginine to tryptophan substitution at amino acid position 1788 of ankyrin-B relative to NCBI database Accession No. GI:119626696;
(h) a serine to proline substitution at amino acid position 1791 of ankyrin-B relative to NCBI database Accession No. GI:119626696;
(i) a glutamic acid to lysine substitution at amino acid position 1813 of ankyrin-B relative to NCBI database Accession No. GI:119626696;
(j) a valine to methionine substitution at amino acid position 1777 of ankyrin-B relative to NCBI database Accession No. GI:119626696;
(k) an arginine to isoleucine substitution at amino acid position 1404 of ankyrin-B relative to NCBI database Accession No. GI:119626696;
(l) a valine to isoleucine substitution at amino acid position 1516 of ankyrin-B relative to NCBI database Accession No. GI:119626696;
(m) a glutamic acid to lysine substitution at amino acid position 1452 of ankyrin-B relative to NCBI database Accession No. GI:119626696;
(n) a serine to threonine substitution at amino acid position 1721 of ankyrin-B relative to NCBI database Accession No. GI:119626696;
(o) a threonine to asparagine substitution at amino acid position 1726 of ankyrin-B relative to NCBI database Accession No. GI:119626696;
(p) a glutamic acid to lysine substitution at amino acid position 1578 of ankyrin-B relative to NCBI database Accession No. GI:119626696; or
(q) any combination of (a) to (p).

In representative embodiments, the subject is African or of African ancestry (e.g., African-American) and the ankB loss of function allele results in (the encoded protein comprises):
(a) an arginine to tryptophan substitution at amino acid position 1450 of ankyrin-B relative to NCBI database Accession No. GI:119626696;
(b) a valine to aspartic acid substitution at amino acid position 1516 of ankyrin-B relative to NCBI database Accession No. GI:119626696;
(c) a threonine to asparagine substitution at amino acid position 1552 of ankyrin-B relative to NCBI database Accession No. GI:119626696;
(d) a leucine to isoleucine substitution at amino acid position 1622 of ankyrin-B relative to NCBI database Accession No. GI:119626696;
(e) a threonine to asparagine substitution at amino acid position 1626 of ankyrin-B relative to NCBI database Accession No. GI:119626696;
(f) a serine to proline substitution at amino acid position 1791 of ankyrin-B relative to NCBI database Accession No. GI:119626696; or
(g) any combination of (a) to (f).

In representative embodiments, the subject is Caucasian (i.e., European or of European ancestry) and the ankB loss of function allele results in (the encoded protein comprises):
(a) a glutamic acid to glycine substitution at amino acid position 1425 of ankyrin-B relative to NCBI database Accession No. GI:119626696;
(b) an arginine to tryptophan substitution at amino acid position 1450 of ankyrin-B relative to NCBI database Accession No. GI:119626696;
(c) a valine to aspartic acid substitution at amino acid position 1516 of ankyrin-B relative to NCBI database Accession No. GI:119626696;
(d) an arginine to tryptophan substitution at amino acid position 1788 of ankyrin-B relative to NCBI database Accession No. GI:119626696;
(e) a glutamic acid to lysine substitution at amino acid position 1813 of ankyrin-B relative to NCBI database Accession No. GI:119626696;
(f) an arginine to isoleucine substitution at amino acid position 1404 of ankyrin-B relative to NCBI database Accession No. GI:119626696;
(g) a valine to isoleucine substitution at amino acid position 1516 of ankyrin-B relative to NCBI database Accession No. GI:119626696;
(h) a glutamic acid to lysine substitution at amino acid position 1452 of ankyrin-B relative to NCBI database Accession No. GI:119626696;
(i) a serine to threonine substitution at amino acid position 1721 of ankyrin-B relative to NCBI database Accession No. GI:119626696;
(j) a threonine to asparagine substitution at amino acid position 1726 of ankyrin-B relative to NCBI database Accession No. GI:119626696;
(k) a glutamic acid to lysine substitution at amino acid position 1578 of ankyrin-B relative to NCBI database Accession No. GI:119626696; or
(l) any combination of (a) to (k).

In representative embodiments, the subject is Hispanic or of Hispanic ancestry and the ankB loss of function allele results in (the encoded protein comprises) an arginine to tryptophan substitution at amino acid position 1788 of ankyrin-B relative to NCBI database Accession No. GI:119626696.

In representative embodiments, the subject is Asian or of Asian ancestry (e.g., Han Chinese or of Han Chinese ancestry) and the ankB loss of function allele results in (the encoded protein comprises) a valine to methionine substitution at amino acid position 1777 of ankyrin-B relative to NCBI database Accession No. GI:119626696;

In embodiments of the invention, a human subject comprises an ankB gene comprising a SNP as shown in Table 2. SNPs in the ank2 gene are also described in Mohler et al., Circulation 115:432-441 (2007).

In representative embodiments, the ankB loss of function allele increases the risk/incidence of type 2 diabetes in one gender to a greater extent than the other. For example, increased risk/incidence of type 2 diabetes in males versus females or vice versa. As one illustration, in embodiments of the invention, the subject is a human male and the ankyrin B loss of function allele results in (comprises) an arginine to tryptophan substitution at amino acid position 1788 of ankyrin-B relative to NCBI database Accession No. GI:119626696.

The ankryin-B protein is conserved across species. Mutations corresponding to the human mutations described herein can be determined by those skilled in the art using known techniques. For example, in the mouse, the ankB gene is located on chromosome 3 (see, e.g., the mouse genomic sequence ENSMUSG00000032826 in the ensemble database), rather than chromosome 4 in the human (see, e.g., the human genomic sequence at ENSG00000145362 in the ensembl database).

As one illustration, a mouse ankryn-B comprising a mutation homologous to the human L16621 mutation described herein can be generated by introducing the following mutation into the ankB coding sequence on mouse chromosome 3:

L1622I mouse: Chr3 (126637532 to 126637473):
AGCCCAGCAGCAGCACTG/ATCTCTCCTCAAATGCACCAGGAGCCAGTTCAACAAGATTTCTCA

Further, a mouse ankryn-B comprising a mutation homologous to the human R1788W mutation described herein can be generated by introducing the following mutation into the ankB coding sequence on mouse chromosome 3:

R1778W mouse: Chr3 (126632792 to 126632733):
ATCATTAGGC/TGGTACGTTTCCTCTGATGGCACAGAGAAGGAGGAGGTTACCATGCAGGGA

The ankB loss of function alleles can be detected by any suitable method. As one non-limiting example, a suitable sample comprising nucleic acid and/or protein from the subject can be obtained and the nucleic acid and/or protein can be prepared therefrom and analyzed according to well-established protocols for the presence and/or absence of one or more ankB loss of function alleles. The presence or absence of a loss of function ankB allele can be determined by any suitable method known in the art including determinations made at the amino acid and/or nucleotide sequence level. For example, the presence or absence of a loss of function ankB allele can be determined by evaluating the amino acid sequence of ankyrin-B (including the full length sequence and/or a portion thereof) produced in the subject and/or by determining the nucleotide sequence of the ankB gene (including the full-length gene and/or a portion thereof) in the subject (e.g., by determining the nucleotide sequence of genomic DNA, cDNA and/or mRNA transcript or a portion of any of the foregoing) in nucleic acid of the subject.

Methods of determining protein sequences are known in the art including but not limited to direct sequencing methods such as mass spectrometry based methods and methods based on the Edman degradation reaction, and indirect methods (i.e., determining the nucleotide sequence of the ankB gene, cDNA, mRNA transcript, etc. or a portion thereof and predicting the protein sequence therefrom).

Methods of determining nucleic acid sequences are also known in the art and include, without limitation, Maxam-Gilbert sequences methods, chain-termination methods, and dye terminator sequencing methods. Optionally, the nucleic acid sequencing method can include an amplification step to amplify all or a portion of the ankB nucleic acid prior to sequencing.

Nucleic acid amplification methods are known in the art and including without limitation polymerase chain reaction, ligase chain reaction, strand displacement amplification, transcription-based amplification, self-sustained sequence replication (3SR), Qβ replicase protocols, nucleic acid sequence-based amplification (NASBA), repair chain reaction (RCR) and boomerang DNA amplification (BDA)). In embodiments of the invention, the amplification product can then be visualized directly in a gel by staining or the product can be detected by hybridization with a detectable probe. When amplification conditions allow for amplification of two or more different alleles, the alleles can be distinguished by a variety of well-known methods, such as hybridization with an allele-specific probe, secondary amplification with allele-specific primers, by restriction endonuclease digestion, by electrophoresis, or by nucleic acid sequencing.

In carrying out the methods of the invention, those skilled in the art will appreciate that the genotype of the subject (e.g., heterozygous or homozygous for the ankB loss of function allele) can be taken into consideration. Optionally, correlations or comparisons are made between heterozygous and/or homozygous subjects for the ankB loss of function allele and subjects that do not have the ankB loss of function allele (or subjects that do not have any ankB loss of function allele), e.g., for determining risk for developing type 2 diabetes, for correlating the presence of the ankB loss of function allele with risk of developing type 2 diabetes, for correlating the effectiveness of a treatment for preventing or treating type 2 diabetes in a subject with an ankB loss of function allele, in prognostic methods, and the like. So, for example, in representative methods of identifying whether a subject is at risk for developing type 2 diabetes, the method comprises determining whether the subject is heterozygous and/or homozygous for an ankB loss of function allele.

Having described the present invention, the same will be explained in greater detail in the following examples, which are included herein for illustration purposes only, and which are not intended to be limiting to the invention.

### Example 1

### Materials & Methods

### Antibodies and Molecular Construct Preparation.

Full length 220 kD human ankyrin-B containing a carboxy terminal FLAG tag was inserted into AdEasy pShuttleCMV (Stratagene) using molecular techniques. Full length 220 kD ankyrin-B containing a carboxy terminal His tag was inserted into BakPak 9 (Clontech), using standard molecular techniques. The R/W mutation was generated using Quikchange Mutagenesis (Stratagene). Constructs were sequenced and expressed in 293K cells to ensure full length protein and FLAG tag integrity. Affinity purified ankyrin-B and G antibodies were generated in rabbits against a bacterially expressed cleaved fusion protein representing the carboxy-terminal domain of the ankyrin. Mouse monoclonal ankyrin-B antibody was generated as described previously (5). Affinity purified pan-InsP3R antibody was generated in rabbits against bacterially expressed cleaved fusion protein representing the C-terminal cytoplasmic domain of InsP3R. Guinea pig anti-insulin, rabbit anti-glucagon, and rabbit anti-somatostatin antibodies (catalog number 180067, 180064, 180078, respectively) were purchased from Invitrogen. Glyceraldehyde-3-phosphate dehydrogenase and M2 recognizing FLAG tag DDDDK epitope monoclonal antibodies (ab8245 and ab49763) were purchased from Abcam. Dihydropyridine receptor (DHPR) antibody (MA1-90408) were purchased from Affinity Bioreagents. KATP channel subunit Kir6.2 and muscarinic receptor 3 antibodies (ab5495 and ab9453) were purchased from Millipore.

### Protein Alignment.

Protein alignments were performed in CLUSTALW using the following protein sequences from NCBI: Homo sapiens gi|119626696|gb|EAX06291.1; Macaca mulatta gi|109075425|ref|XP_{—}001095471.1; Canis familiaris gi|74002173|ref|XP_{—}545031.2; Mus musculus gi|37590265|gb|AAH59251.1|; Rattus norvegicus gi|109467596|ref|XP_{—}001076082.11; Pan troglodytes gi|114595754|ref|XP_{—}517403.2; Gallus gallus gi|118090374|ref|XP_{—}420641.2.

Genetic Studies. ANK2 variants reported previously to have severe functional consequences in cardiomyocytes were used for SNP analysis of 1122 patient samples from the GENNID collection. Genomic DNA was purchased from Corriell Laboratories. SNP genotyping was performed using the ABI 7900HT Taqman SNP genotyping system (Applied Biosystems, Foster City, Calif., United States), which uses a PCR-based allelic discrimination assay in a 384-well-plate format with a dual laser scanner. Allelic discrimination assays were purchased from Applied Biosystems, or, if the assays were not available, primer and probe sets were designed and purchased through Integrated DNA Technologies (Coralville, Successful genotyping was obtained for greater than 95% of the DNA samples used in the study. Patient partial pedigree information, diabetes status, race, age, sex, BMI, glucose and lipid levels, and history of heart and kidney disease were available in the Corriell GENNID catalog. P values for association were determined using chi-squared analysis for diabetes status, sex, and history of heart or kidney disease. For comparisons of numeric values, including BMI, age, fasting glucose, and lipid levels, p values were determined using a two-tailed T-test and p values less that 0.05 were considered significant.

Animal care. AnkB mice were backcrossed >20 generations (>99.5% pure) into a C57/BI6 background before experiments. AnkB(+/+), (+/−), and (−/−) mice were housed 4-5 per cage in the same barrier facility with temperature and humidity and 12 hour light/dark cycles controlled. The mice were fed standard mouse chow (Lab Diet, 23% protein, 4.5% fat, 6.0% fiber, 8.0% ash, 2.5% minerals (0.95% Ca2+, 0.67% phosphorus, 0.40% non-phytate phosphorus), 56% complex carbohydrate from overhead wire feeders) and water ad libitum.

### In Vivo Physiological Studies.

Glucose tolerance tests: Oral GTT and IGTT were performed on 4-6 month old mice subjected to an overnight (12 h) fast. For the oral GTT, glucose (2 mg/g) was administered via oral gavage after being anesthetized with isofluorane gas. For the IGTT, mice received glucose (2 mg/g) via intraperitoneal injection. For both tests, blood samples were collected from the tail vein before (0 min) and time intervals thereafter (5, 10, 15, 30, 60, 120 min). For fasted/fed serum insulin and Glp-1 measurements, blood was collected from the submandibular vein before (0 min) or 30 minutes after oral or i.p. glucose administration. Data presented represent the mean blood glucose level+/−SEM for each time point. Glucose measurements were performed using a handheld automated glucometer (Accucheck). Significance for the tolerance tests was determined by two way ANKOVA. Area under the curve (AUC) calculations were performed using the trapezoidal rule and significance was determined by two-tailed T test. Serum insulin was measured by insulin ELISA (Crystal Chem Inc). Active serum Glp-1 was measured using MULTIARRAY (Mesoscale Discovery).

Insulin tolerance tests: Using 4-6 month littermates, overnight fasted mice were injected with recombinant human insulin (Sigma, 0.75 U/kg). Blood glucose was monitored before (0 min) and at time intervals (15, 30, 60 min) after insulin injection by tail vein blood collection. Data represent the mean blood glucose value+/−SEM. Mouse weights. Mouse weights were determined on 4-6 month old mice, 10 animals/genotype. Measurements were taken three times on each animal and averaged. Data represents the mean weight (g)+/−SEM.

### Islet Morphometric Analysis.

Pancreases from 4-6 month old ankB(+/+ and (+/−) mice were used for immunofluorescent detection of the islet maker insulin as described in the following section. Six animals/genotype were used. Islet density (number islets/section) were determined for all samples. Islet size was determined using LSM 510 software. Total insulin content was determined using acid ethanol extraction as described previously. All data represent the mean value+/−SEM. P value was calculated using a two-tailed T test.

### Immunofluorescence.

Neonatal or 16-24 week mouse pancreases were washed with phosphate buffered saline (PBS, pH 7.4) and fixed in cold 4% paraformaldehyde (4° C.). Pancreases were embedded in paraffin and 5 micrometer sections were mounted on glass slides and stored at room temperature. Sections were rehydrated and permeabilized before use by incubating in xylenes for 5 minutes for two washes, 100% ethanol for 2 minutes for two washes, followed by one 1 minute wash each of 95%, 90%, 80%, and 70% ethanol. Slides were then incubated in deionized water 10 minutes, followed by 2 five minute washes in 1×PBS. Sections were then incubated in blocking buffer for 30 minutes (PBS containing 1% BSA, 1% fish oil gelatin, 5% horse serum, and 0.02% Tween-20) and in primary antisera overnight at 4° C. Following washes (PBS plus 0.025% Tween-20), cells were incubated in secondary antisera (Alexa 488, 568; Molecular Probes) for 2-3 hours at 4° C. and mounted using Vectashield (Vector) and #1 coverslips. Images were collected on a Zeiss LSM 510 confocal microscope using a 40 power oil objective, pinhole equals 1.0 Airy Disc) using Carl Zeiss Imaging software. Both channels were collected on PMTS. Images were imported into Adobe Photoshop for cropping and contrast adjustment. Image quantitation was performed using LSM-Image Examiner software, histogram function. Equivalent size regions of interest were marked on islets and background regions, and intensity-background was averaged. Values represent the mean+/−standard error of the mean (SEM).

### Preparation and Use of Recombinant Adenoviruses.

INS-1-derived cell line 823/3 was cultured as described previously (28). Small interfering RNA (siRNA) sequences corresponding to rat ankyrin-B siRNA1: GGCCAGAAGATCTCAAGGA (SEQ ID NO:2), siRNA2: GCTGTGTAGCATTTTAACA (SEQ ID NO:3), or a control siRNA which is siRNA 1 mutated at three base sites: GGCCCGAAGAGCTCAAGGA (SEQ ID NO:4), were cloned into vector EHOO6 and used for construction of Ad-ankB siRNA recombinant adenoviruses by the methods described (29). Complementary DNAs encoding human ankyrin-B or ankyrin-B W/W (7, 8) were used to prepare recombinant adenoviruses (AdCMV-h ankB and h ankB RAN) using the AdEasy system (Stratagene catalog number 240010). An adenovirus containing the green fluorescent protein (GFP) gene (AdCMV-_GFP) was used as a control. Purified viruses were incubated with INS-1 823/3 cells or islets at multiplicities of infection (MOI) of 20-50 for 18 h. Assays were undertaken 72 h later.

### Islet Isolation and Insulin Secretion Assays.

Islets were isolated from ankB littermates and male Wistar rats by pancreatic perifusion as previously described (28). Islets were maintained in culture medium containing 11 mM glucose until the day of the assay. Insulin secretion was assayed in HEPES balanced salt solution (HBSS) (114 mM NaCl, 4.7 mM KCl, 1.2 mM KH₂PO₄, 1.16 mM MgSO₄, 20 mM HEPES, 2.5 mM CaCl₂, 25.5 mM NaHCO₃, and 0.2% BSA, pH 7.2). Islets were pre-incubated HBSS containing 3 mM glucose for 2 hours. Insulin secretion was then measured by using static incubation for a 1 h period in HBSS containing 3 mM glucose. Islets were then transferred to HBSS containing 16.7 or 8 mM glucose for 1 hour, and then HBSS containing 16.7 or 8 mM glucose plus 0.1 mM carbachol or 100 nM Glp-1 for 1 hour. Following the incubations, islet samples were normalized for insulin content by extraction with 1 M acetic acid in 0.1% BSA. Static incubation samples and extract samples were analyzed for insulin concentrations via radioimmunoassay with the insulin Coat-a-Count kit (Diagnostic Products, Los Angeles). Values presented represent the mean values+/−SEM.

Measurements of Dynamic Insulin Release from Isolated Islets.

Before the perifusion, islets were preincubated in a solution (buffer A) containing 25 mM N-(2-Hydroxyethyl) piperazine-N′(2-ethanesulfonic acid) (HEPES), pH 7.4, 125 mM NaCl, 5.9 mM KCl, 1.28 mM CaCl₂, 1.2 mM MgCl₂, 0.1% BSA, and 3 mM glucose for 60 min at 37° C. The assay buffer A and the stimuli were perfused through a sample container harboring 100 islets immobilized in Bio-Gel P-4 polyacrylamide beads (BioRad) at 37° C. The flow rate was 90 μL/minute and the perifusate fractions were collected every 2 minutes. Insulin measurements of the samples were performed by a microsphere-based two-photon excitation fluorometer (TPX-technology; ArcDia Diagnostics, Turku, Finland) using a human insulin standard (Sigma-Aldrich).

### Intraislet Calcium Measurements Using Fura-2.

Islets from 8-10 month ankB(+/+) and (+/−) mice were isolated as described above and incubated overnight in medium containing 11 mM glucose. The following day, islets were washed with perfusion buffer (140 mM NaCl, 5.9 mM KCl, 2.56 mM CaCl2, 1.2 mM MgCl2, 1 mM bovine serum albumin, and 25 mM HEPES, pH7.4) and transferred to perfusion buffer containing 3 mM glucose and 2 uM Fura-2 AM (Invitrogen). Islets were incubated 45 min at 37° C. Islets were then affixed to small open perifusion chamber (volume 150 μL) with a coverslip bottom using Puramatrix Peptide Hydrigel (BD Biosciences). Chamber was then mounted on a Zeiss Axiovert epifluorescence inverted microscope fitted with a Plan-Neofluar 16x/0.50 objective. The fluorescence (excitation at 355 or 380 nm) was recorded by a slow-scan charged-coupled device (CCD) camera (Andor Technology) and quantitated using AndorIQ software. All perifusions were performed at 37° C. using perfusion buffer containing either 3 mM glucose, 11 mM glucose, 30 mM KCl in 3 mM glucose, or 0.1 mM carbachol in 3 mM glucose. To assay carbachol effects in the absence of calcium, buffer containing 2 mM EGTA and 0 mM Ca²⁺ was used. Data represent the mean+/−SEM of 3-7 individual islet recordings/animal using 6 animals/genotype. Significance was calculated using two-tailed T test.

### Real Time PCR Studies.

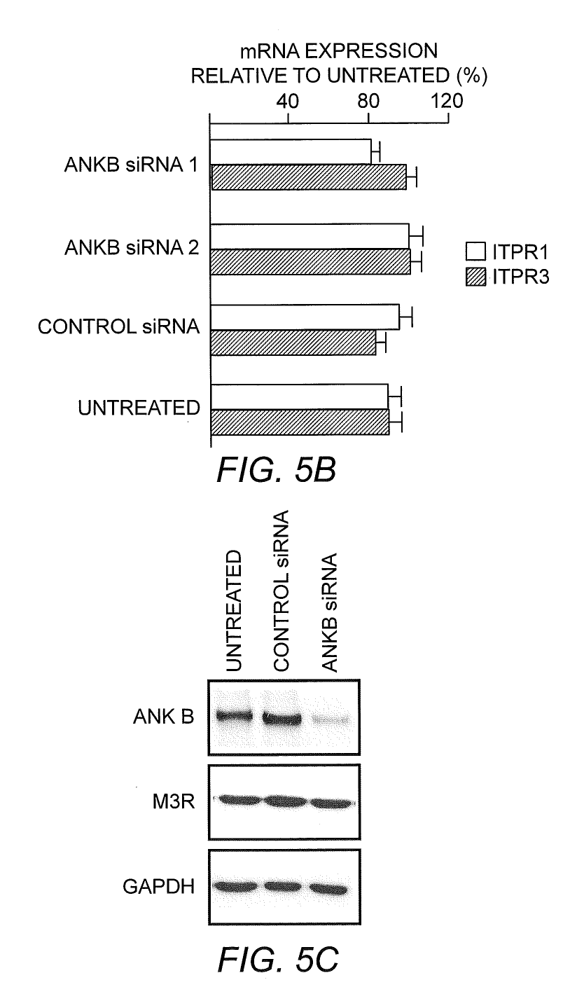

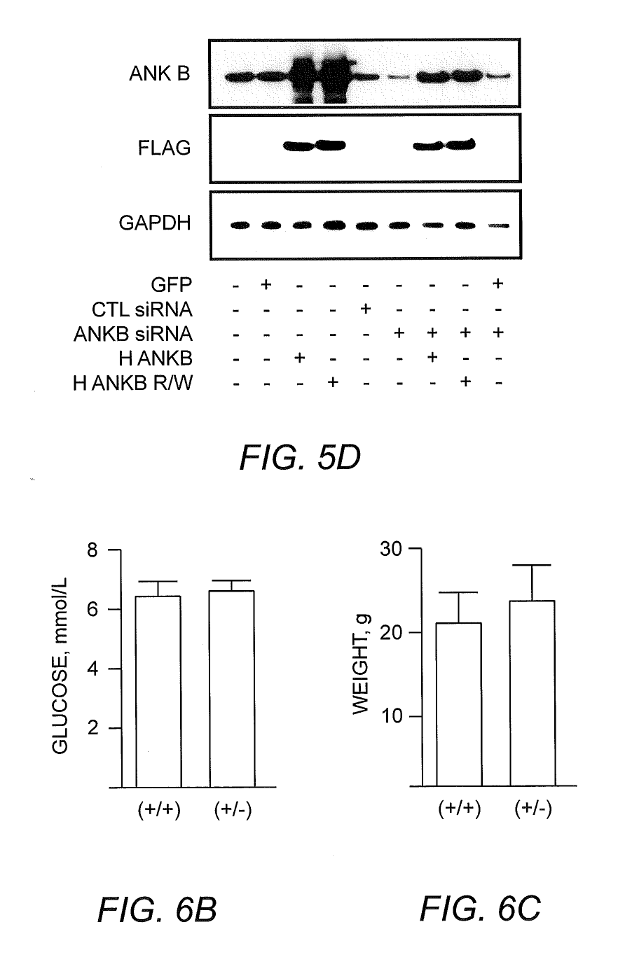

Total RNA was isolated from INS-1 823/3 cells treated with ankyrin-B specific, control, or no siRNA and converted to cDNA using Applied Biosystems Cells to CT kit. Gene expression levels for InsP3R genes ITPR1-3 were measured by real time quantitative PCR (7500 SDS, Applied Biosystems). GAPDH expression served as an internal control. Reactions were carried out in triplicate. Data are represented as fold expression relative to ITPR1 (FIG. 5A) or relative to untreated (FIG. 5B). Data represent the mean+/−SEM.

### Immunoblotting and Protein Sample Preparation.

Lysates from INS-1 cells and islets were prepared from cell pellets washed with 1×PBS, dissolved in RIPA buffer and sonicated. Samples were normalized for protein content using the BCA protein assay kit (Pierce Biotechnology) and subjected to polyacrylamide gel electrophoresis using NuPAGE (Invitrogen) 3-8% Tris-acetate gels (Invitrogen). Gels were transferred to PVDF membrane for western blot analysis using the antibodies specified. Membranes were blocked in PBST containing 5% milk for 30 minutes and incubated in primary antibody overnight. The following day, the membranes were washed in PBST and incubated with HRP-conjugated secondary antibody for 2-3 hours at 4° C. Blots were then washed and developed using ECL (Pierce Biotechnology). Bands were detected by film autoradiography and quantified using densitometry software. Values represent the mean+/−standard error of the mean (SEM).

### Protein Turnover Measurements.

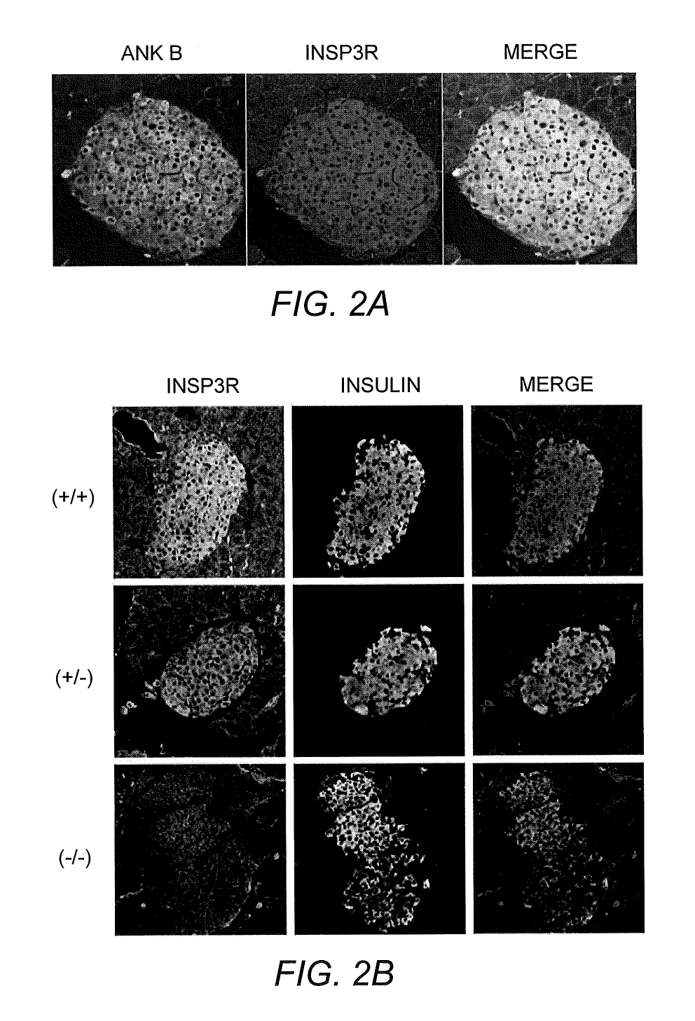

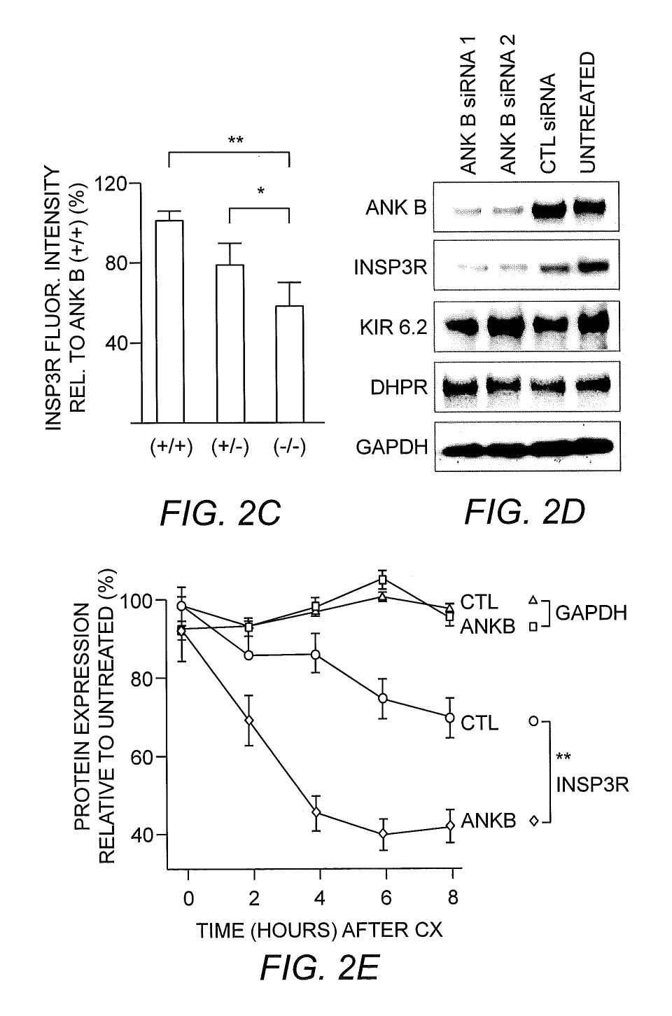

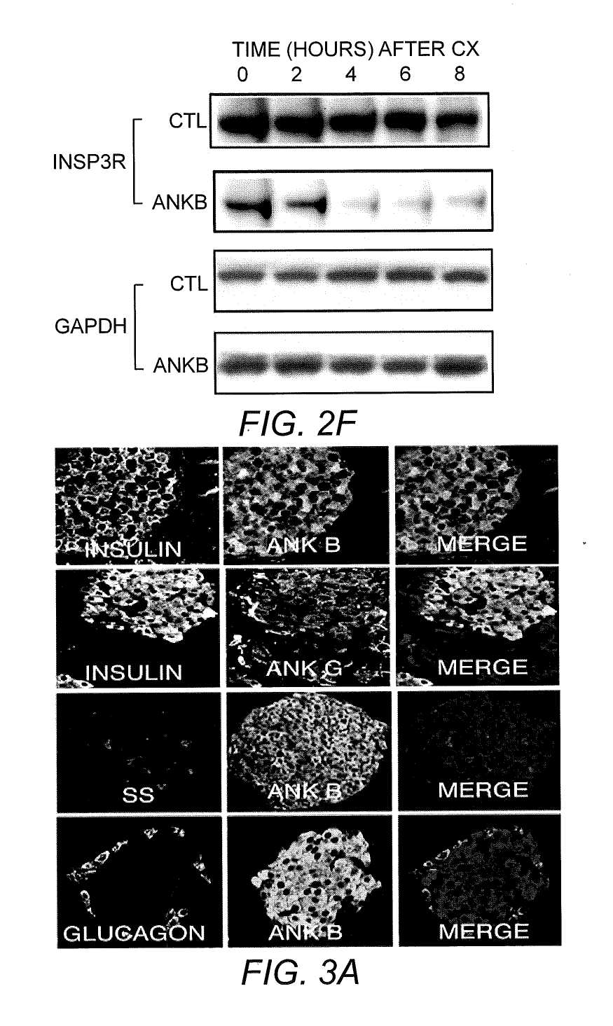

Ins-1 823/3 cells grown in 12 well plates were treated with ankyrin-B specific or control siRNA and grown to confluency (1×10⁶ cells/well) were incubated with 1 uM cycloheximide (Cx, Sigma) to inhibit protein synthesis. After 30 min, cells were washed with 1×PBS and fresh medium was added. Cell lysates were prepared for each well in duplicate before cycloheximide administration (0 h) and at time intervals thereafter (2, 4, 6, 8 h). InsP3R and GAPDH protein levels in lysates were measured by immunoblot and were quantified by blot densitometry. Data (FIG. 2E) represent the mean protein levels+/−SEM. Significance was determined by two way ANKOVA.

### InsP3R/220 kD Human Ankyrin-B and Ankyrin-B Membrane-Binding Domain Purification.

220 kD Histidine-tagged ankyrin-B and ankyrin-B R/W were expressed using the BakPak baculovirus expression system (Clontech). The proteins were purified on an NiNTA affinity column (GE). InsP3R was purified from bovine brain cerebellum as described previously (30). Protein G-conjugated Dynabeads were purchased from Dynal Biotech. Ankyrin-B membrane-binding domain (MBD) with the addition of the first 80 residues of the spectrin-binding domain (SBD) containing a monoclonal antibody epitope was expressed in bacteria and purified as described previously (6).

### In Vitro Binding Experiments.

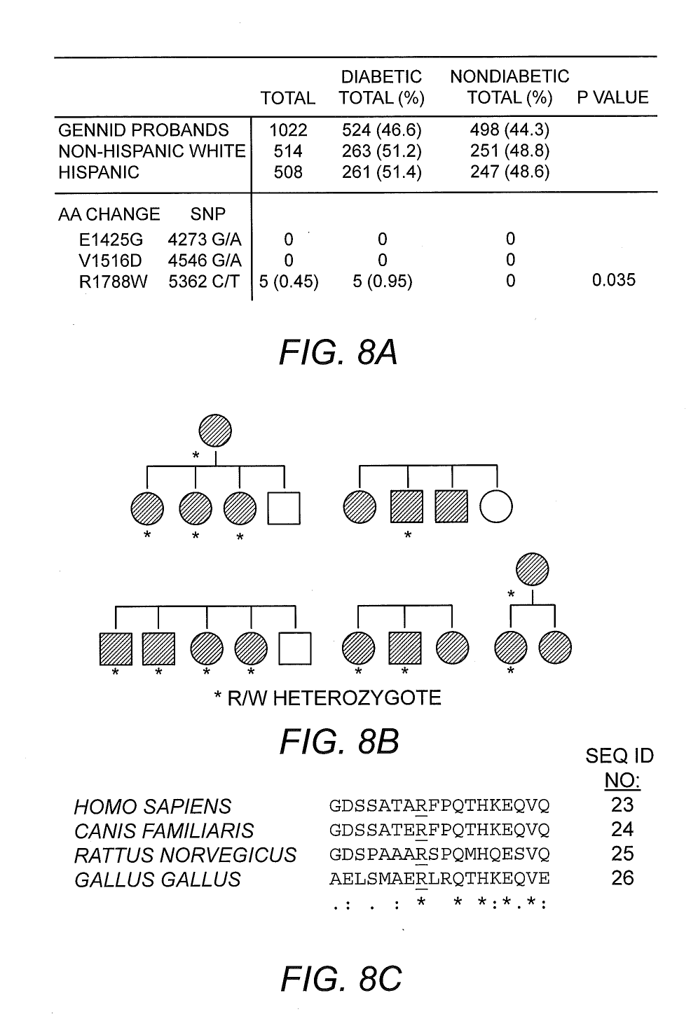

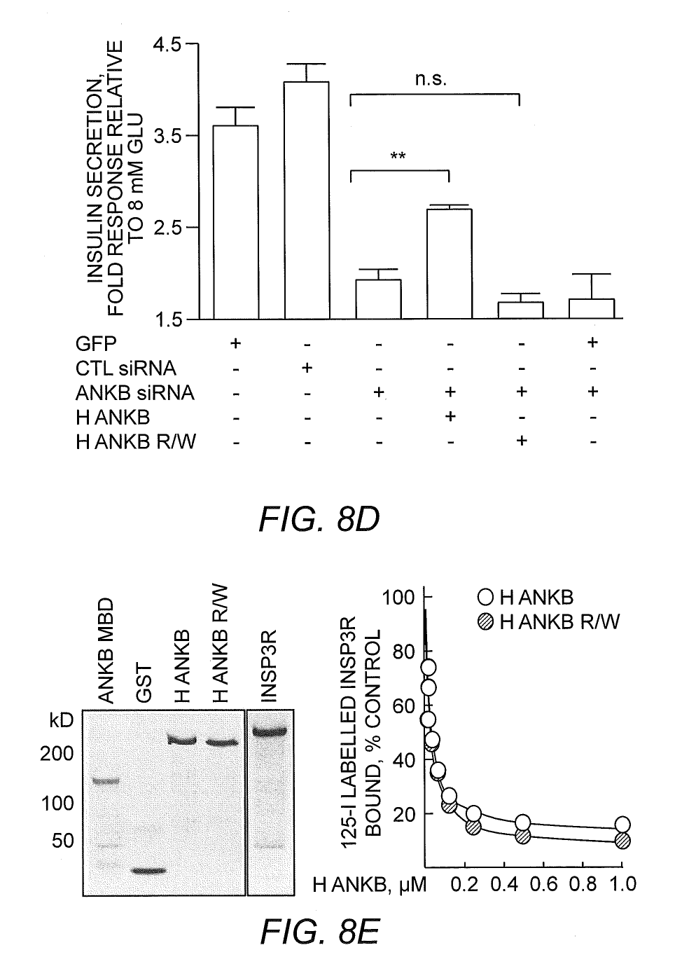

Glutathione-conjugated sepharose beads (Invitrogen) were loaded with either 0.1 uM GST conjugated ankyrin-B membrane-binding domain (MBD) or GST alone. Ankyrin-B beads were incubated with ¹²⁵I-labelled purified cerebellar InsP3R and increasing concentrations of purified full length human ankyrin-B or ankyrin-B R/W (0-1 uM) in binding buffer (20 mM Hepes, 50 mM NaCl, 1 mM EDTA, 1 mM NaN3, 0.2% Triton X-100; pH 7.3) for two hours at 4° C. in a final volume of 50 μl. Beads were layered over 20% glycerol barriers and spun in Beckman J6B centrifuge at 4000 RPMs. Samples were then frozen on dry ice, pellets cut off and assayed for ¹²⁵I in a gamma counter. For Scatchard plot (FIG. 8E, right panel), values for non-specific binding were determined using GST alone beads and were subtracted. Left panel shows a PAGE gel stained for protein with Coomassie Blue. Purified ankyrin-B MBD-GST (lane 1); GST alone (lane 2); full length human ankyrin-B (lane 3); human ankyrin-B containing RAN mutation (lane 4); purified cerebellar InsP3R (lane 5).

### Example 2

### InsP₃R Expression Levels ankB(+/+) and ankB(−/−) Mice

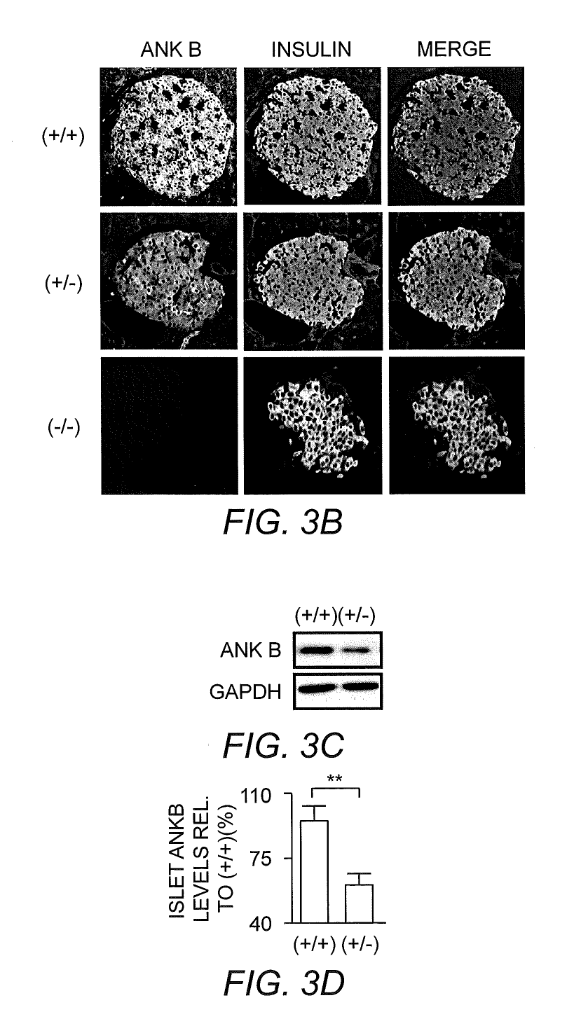

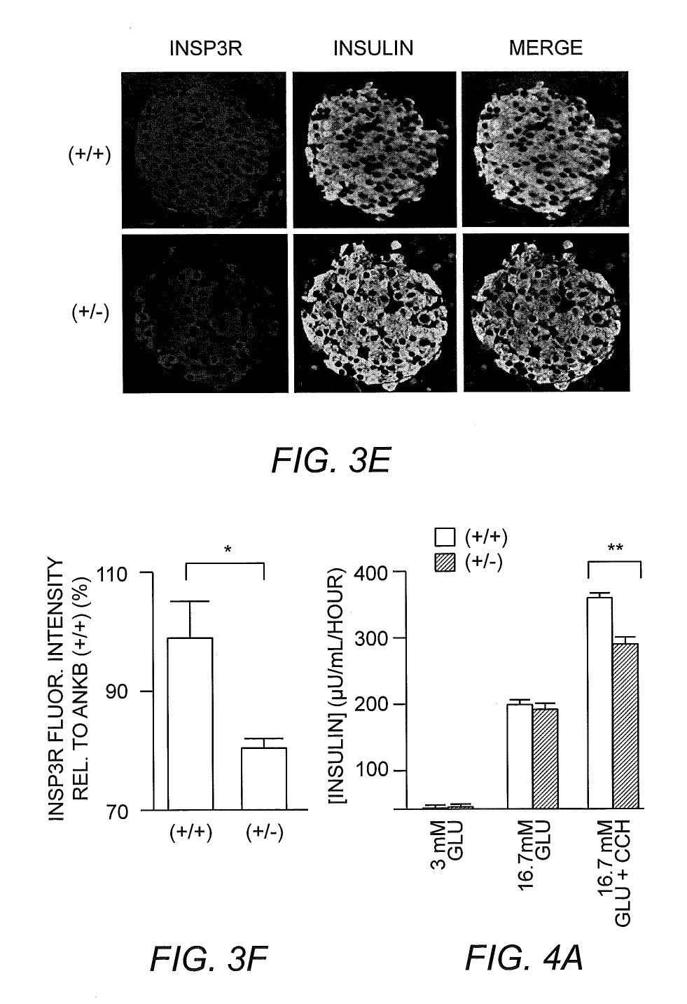

Ankyrin-B is enriched specifically in insulin-secreting beta cells of the endocrine pancreas and is absent from cells secreting either glucagon or somatostatin ((9); FIG. 2A; FIG. 3A). Moreover, ankyrin-B exhibits an identical staining pattern to the InsP₃R receptor within pancreatic islets (FIG. 2A). We next evaluated whether InsP₃R expression levels were altered in mice heterozygous or homozygous for a null mutation in the gene encoding ankyrin-B. Of these, only ankB(+/−) mice reach adulthood. AnkB(+/−) mice are haploinsufficient, with islet ankyrin-B expression being ˜50% wild type levels. ((9), FIG. 3 B,C,D). As ankB(−/−) mice die perinatally, we first compared InsP₃R levels in neonatal ankB(+/+), (+/−), and (−/−) pancreatic islets by immunofluorescence (FIG. 2 B,C). Whereas neonatal ankB(+/−) islets demonstrated a 21% reduction in InsP3R fluorescence intensity (p=0.05,n=6), ankB(−/−) islets demonstrated a 42% reduction (p=0.03, n=6) as compared to ankB(+/+) islets. Pancreatic islets of adult ankB (+/−) mice exhibit a comparable reduction in InsPR intensity of 18% (p=0.03, n=6, FIG. 3 E,F).

To study how ankyrin-B affects InsP₃R protein levels, we evaluated both InsP₃R mRNA and protein expression during ankyrin-B knockdown using the rat insulinoma cell line INS-1 823/3. Quantitative PCR showed no difference in InsP₃R mRNA expression in the ankyrin-B siRNA treated cells compared to untreated control (FIG. 5 A,B). By contrast, immunoblot analysis of protein expression during ankyrin-B knockdown showed a dramatic reduction in InsP₃R levels (FIG. 2 D). Other proteins known to be essential to glucose-stimulated insulin secretion, including the dihydropyridine receptor (DHPR) and the K_{ATP} channel subunit Kir6.2 were unaffected. The reduction in InsP₃R protein expression in the context of normal mRNA expression suggests that the stability of InsP₃R might be reduced. We next measured InsP₃R protein turnover by blocking protein synthesis with cycloheximide (1 uM) and quantifying IP3R expression levels thereafter (FIG. 2 E,F). After 8 hours, InsP₃R expression was reduced by 57% in cells treated with ankyrin-B siRNA and 22% in those treated with control siRNA (p=0.01, n=4). The increase in protein turnover indicates that ankyrin-B promotes InsP₃R stability.

### Example 3

### Ankyrin-B is Essential for Normal Parasympathetic Augmentation of Glucose Stimulated Insulin Secretion

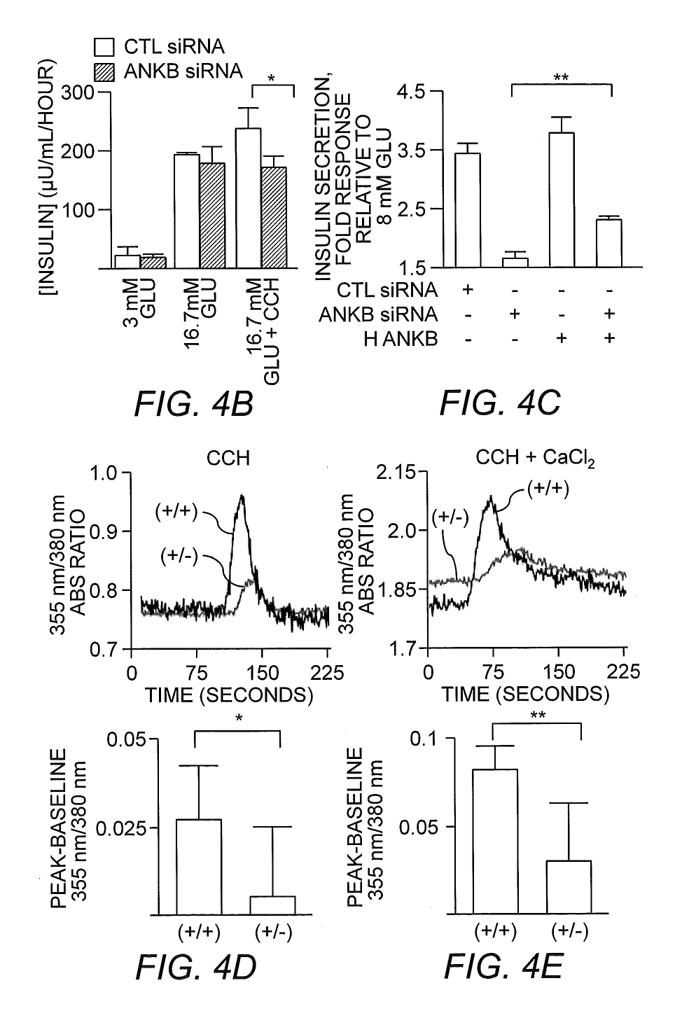

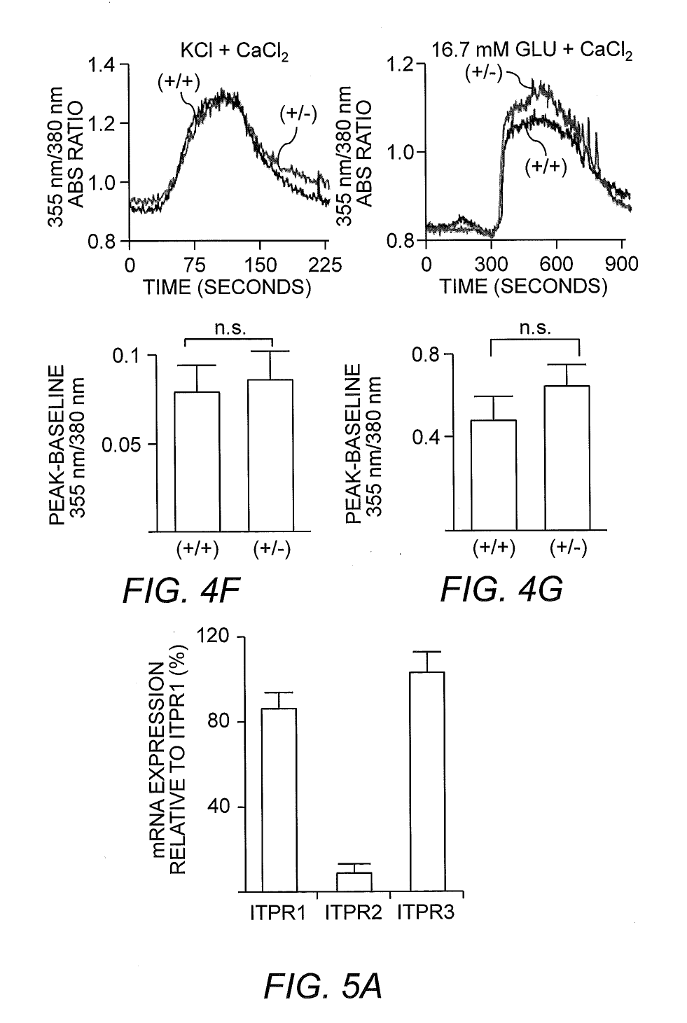

We next evaluated ankyrin-B's role in pancreatic islet function. Since the InsP₃R plays an essential role in the beta cell response to cholinergic stimuli (10, 11), we evaluated whether a reduction of ankyrin-B, and therefore InsP3R, would affect acetylcholine-mediated insulin release. As treatment of islets with the muscarinic receptor agonist carbachol leads to significant potentiation of insulin release in the presence of stimulatory glucose concentrations (12-14), we compared insulin release from islets isolated from ankB (+/+) and (+/−) mice and treated with basal glucose (3 mM), stimulatory glucose (11 mM), and stimulatory glucose plus carbachol (0.1 mM). In wild type islets, we found that carbachol potentiated insulin release by 2.1-fold (FIG. 4 A). AnkB(+/−) mouse islets also showed normal insulin release in response to high glucose alone (21.2-fold low glucose levels). However, ankB(+/−) islets demonstrated a 40% reduction in carbachol-stimulated insulin release (p=0.007, n=6).

To confirm that the results we observed in ankB(+/−) islets were specifically due to deficiency in ankyrin-B within the islet rather than other tissues, we measured carbachol-stimulated insulin release in wild type rat islets subjected to adenovirally delivered siRNA. Using this technique, we achieved ˜60% suppression of ankyrin-B protein levels estimated by immunoblots (FIG. 2 D). Importantly, muscarinic receptor protein expression levels were not affected by ankyrin-B knockdown (FIG. 5 C). Similar to our results with islets isolated from haploinsufficient mice, we observed normal glucose stimulated insulin release and an 88% reduction in carbachol-stimulated insulin release (p=0.04, n=6, FIG. 4 B). FIG. 4 C shows that the adenoviral delivery of full-length human ankyrin-B to the knockdown islets leads to partial rescue (−40%) of the phenotype of impaired carbachol-mediated insulin release (p=0.03, n=6). Overall, these data demonstrate that carbachol-dependent insulin release in isolated islets is specifically reduced in the context of ankyrin-B deficiency, and can be partially rescued by expression of exogenous ankyrin-B.

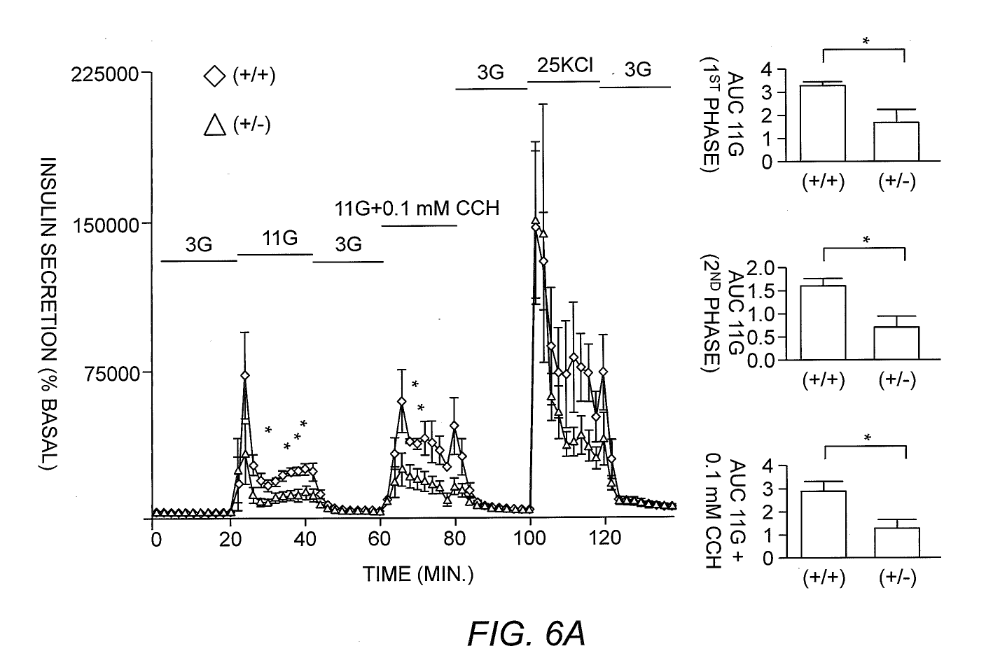

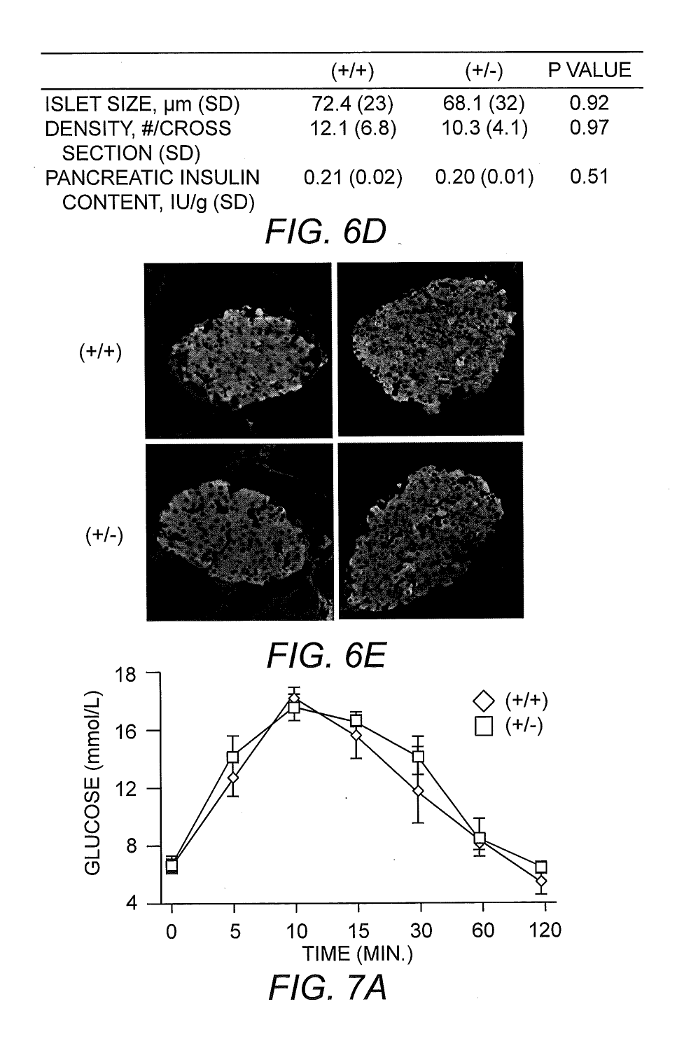

Using islet perifusion, we next explored the time dependency of ankyrin-B's effects on insulin release from islets isolated from ankB(+/−) and (+/+) mice (FIG. 6A). We confirmed that ankyrin-B deficiency reduced carbachol-stimulated insulin release over time (p=0.05, n=3). Using this more sensitive technique, ankB(+/−) mouse islets also showed a reduction in glucose-stimulate insulin release in response to high glucose alone in the first and second phases of insulin secretion (# and # fold, respectively, p=0.05), suggesting that ankyrin-B may also have an some effect on glucose stimulated insulin secretion, as well.

In order to determine how ankyrin-B-deficiency impairs carbachol-mediated insulin release, we next measured intracellular Ca²⁺ dynamics in islets from ankB(+/−) mice. Islets perifused with basal glucose (3.3 mM) produce a transient spike in intracellular Ca²⁺ concentration upon the addition of either 0.1 mM acetylcholine or 0.1 mM carbachol (14-16). This Ca²⁺ spike is observed in the absence of extracellular Ca²⁺, indicating that carbachol stimulates Ca²⁺ release from intracellular (ER) stores. To investigate whether this intracellular Ca²⁺ release is disrupted by ankyrin-B haploinsufficiency, we loaded mouse islets with the fluorescent Ca²⁺ probe Fura-2 and perifused with Ca²⁺-free buffer containing carbachol (0.1 mM). When compared with ankB(+/+) islets, ankB(+/−) islets displayed a ˜79% reduction in intracellular Ca²⁺ release (p=0.01,n=6, FIG. 4 D). Blunted Ca²⁺ release was similarly observed when ankB(+/−) islets were exposed to carbachol (0.1 mM) in buffer containing 5 mM Ca²⁺ (64% reduction, p=0.002, n=6) (FIG. 4 E). To ensure that intracellular Ca²⁺ handling was not grossly affected, we also tested intra-islet Ca²⁺ levels following treatments with KCl and glucose, agents known to depolarize the p-cell plasma membrane and elicit extracellular Ca²⁺ entry via voltage-gated Ca²⁺-channels (17). In ankB(+/−) islets, Ca²⁺ entry was unaffected in response to either of these agents (FIG. 4 F,G). Collectively, these results indicate that ankB(+/−) mouse islets have an impaired ability to release Ca²⁺ from internal stores in response to cholinergic stimuli, which is consistent with the observed reduction in carbachol-mediated insulin release.

### Example 4

### Effects of Ankyrin-B on Glucose Homeostasis In Vivo

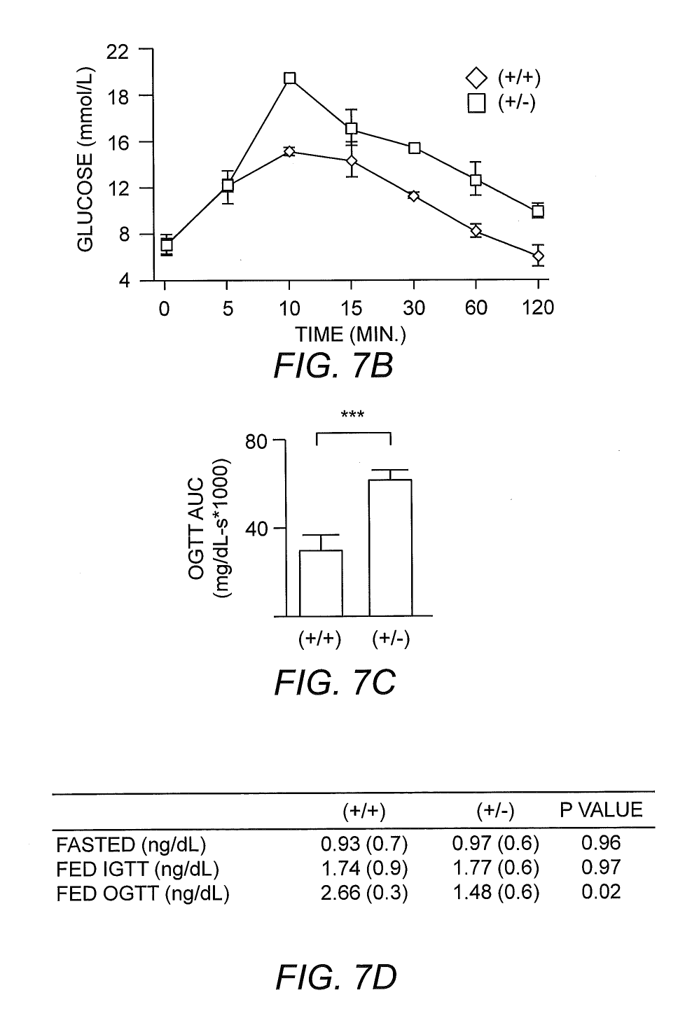

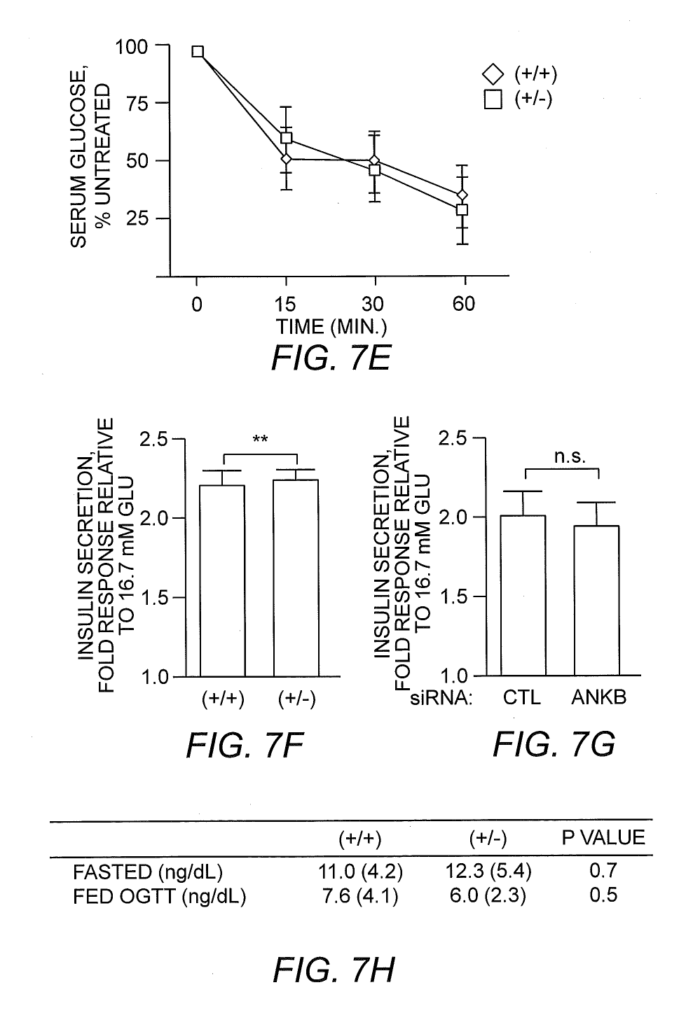

Having demonstrated that ankyrin-B is essential for normal parasympathetic augmentation of glucose stimulated insulin secretion in vitro, we next sought to establish the effects of ankyrin-B on glucose homeostasis in vivo. Mice used in these metabolic studies were litter-matched males, 3-6 months of age with equivalent weights. We administered 2.0 mg/g glucose either intraperitoneally (intraperitoneal glucose tolerance test or IPGTT) or orally (oral glucose tolerance test or oral GTT) and then monitored blood glucose levels over time (FIG. 7 A,B). In contrast to the IPGTT, which relies exclusively upon the absorption of glucose from the peritoneal cavity to stimulate insulin secretion, the OGTT requires glucose to first pass through the gastrointestinal system, thereby allowing parasympathetic stimulation to augment the islet's response to a given glycemic load. Following intraperitoneal injection of glucose, ankB (+/−) and (+/+) mice had identical blood glucose levels as a function of time. Consistent with our in vitro experiments, however, ankB(+/−) mice exhibited impaired tolerance to orally administered glucose as compared with (+/+) controls. Though fasting glucose levels were unaffected (FIG. 7 B, FIG. 6 B), ankB(+/−) mice had elevated plasma glucose levels at 10 minutes and subsequent time points after the glucose challenge (p=0.003, n=9). The area under the curve for oral GTT was increased 32% in ankB(+/−) mice compared to controls (p=0.001, n=9, FIG. 7 C), indicating that ankyrin-B-deficiency disrupts normal postprandial glucose regulation. However, these mice cleared glucose normally when it was injected intraperitoneally. Serum insulin measurements before and 30 min after IP versus oral glucose administration show that ankB(+/−) mice secrete 54% less insulin than their ankB(+/+) counterparts in response to an oral glucose stimulus (p=0.02,n=6, FIG. 7 D). To determine whether altered insulin sensitivity of target tissue influenced these results, we next measured insulin tolerance by administering insulin (1.0 U/kg) intraperitoneally, and then following blood glucose levels (FIG. 7 E). Insulin tolerance was unaffected. Similarly, body weight and morphometric analysis of islets, including size and density, and total pancreatic insulin content were not significantly different in the ankB(+/−) animals (FIG. 6 C,D,E). Impaired oral GTT combined with normal IPGTT indicate that ankB(+/−) mice have normal glucose-stimulated insulin secretion but an inadequate ability to potentiate insulin release by vagal stimulation, consistent with our in vivo islet experiments.

Incretin hormones such as glucagon-like peptide 1 (Glp-1) and gastric inhibitory peptide (GIP) also influence oral glucose tolerance by potentiating glucose stimulated insulin secretion (18). To test whether ankyrin-B (+/−) mice demonstrated normal Glp-1 activity, we measured Glp-1-stimulated insulin release in islets from (+/−) and (+/+) mice (FIG. 7 F). Glp-1 (100 nM) potentiated glucose-stimulated insulin release ˜1.6 fold in wild type animals. AnkB(+/−) islets exhibited no loss of response to Glp-1 and actually demonstrated an increase in insulin release with this peptide (2.1 fold, p=0.01, n=6). In islets treated with ankyrin-B siRNA, Glp-1 mediated insulin release was indistinguishable from the control siRNA treated islets (FIG. 7 G). This suggested that the increase in Glp-1-stimulated insulin secretion in ankB(+/−) mouse islets was not directly caused by islet ankyrin-B deficiency. We then explored the possibility that the increased insulin response to Glp-1 observed in ankB(+/−) islets could be due to enhanced islet sensitivity to Glp-1 in the face of impaired Glp-1 secretion in the ankB(+/−) mice. We measured Glp-1 release during oral GTT in ankB mice (FIG. 7 H). Glp-1 levels were equivalent in (+/−) and (+/+) mice. We also evaluated whether GIP release might be impaired in (+/−) animals (FIG. 7 H). By measuring GIP release during oral GTT, we found that levels of GIP were not decreased, and even may be slightly increased in ankB(+/−) mice, although this trend did not reach significance. The increased Glp-1 sensitivity of ankB(+/−) islets and possible increased release of GIP during meal intake may represent compensation mechanisms for their impaired cholinergic response.

### Example 5

### Identification of AnkB Mutations in Diabetic Subjects

Acetylcholine affects the first phase of insulin secretion, the period that is most often affected in humans with impaired glucose tolerance (19-21). We therefore asked whether ankyrin-B loss of function of mutations are associated with diabetes using the American Diabetes Association GENNID cohort, a collection of partial sibships from families with noninsulin-dependent diabetes (22). We genotyped 524 diabetic probands and 498 non-diabetic controls for the three ankyrin-B mutations previously shown in neonatal cardiomyocytes to have severe loss of function phenotypes: E1425G, V1516D and R1788W (23). The degree to which individual mutations disrupt InsP₃R targeting in neonatal cardiomyocytes roughly correlates with phenotype severity in patients with ankyrin-B syndrome associated arrhythmias (7, 8). In this study, we limited our analysis to Caucasians and Hispanics. The R1788W point mutation, corresponding to a C/T mutation in exon 45 of the ankB (ank2) gene, was found exclusively in patients with diabetes (p=0.035) (FIG. 8 A). In addition, we identified 7 family members of the 5 probands also heterozygous for the R1788W mutation, all of whom were diabetic (FIG. 8 B). By contrast, other factors that can influence diabetic risk, including age, sex and body-mass index, were indistinguishable between patients with the R1788W mutation and the controls (Table 1). Individuals possessing E1425G or V1516D mutations were not identified in this data set. Arginine 1788 is highly conserved amongst species (FIG. 8 C). Moreover, the R1788W mutation modulates ankyrin-B affinity to binding partners, such as obscurin (24) and hsp40 (8). Previously reported minor allele frequencies (MAFs) for R1788W have ranged from 0.09%, in a study of 1152 European centenarians and controls (9), and 0.3% in a study of 664 American cardiac arrhythmia patients (8). The ˜1% MAF in diabetics from our study suggests that the mutation is enriched in this population.

Since the R1788W mutation was present only in type 2 diabetics in the association study, we next tested the ability of human ankyrin-B R1788W to rescue islet cholinergic function during ankyrin-B knockdown (FIG. 8 D). Compared to wild type ankyrin-B, the R1788W variant did not rescue carbachol mediated insulin secretion (p=0.004, n=6). Ankyrin-B R1788W thus demonstrates loss of activity in a cellular assay of islet function. As this phenotype could result from altered binding affinity of InsP₃R for ankyrin-B, we tested the ability of native InsP₃R purified from brain to associate with full-length ankyrin-B containing the R1788W mutation (FIG. 8 E). In this binding assay, InsP₃R demonstrated a normal affinity for R1788W ankyrin-B. This suggests that the impaired carbachol-mediated insulin release is not due to an impaired ability of R1788W ankyrin-B to bind to InsP₃R. Alternatively, R1788W ankyrin-B may disrupt InsP3R targeting to microdomains within pancreatic beta cells in a manner similar to that seen in cardiomyocytes. (5). While localized Ca²⁺ release events have been observed previously in islets and isolated beta cells (25), the millisecond timescale of these events and the small size of the beta cell do not permit sufficient intracellular spatial resolution to study microdomains directly.

We have demonstrated that ankyrin-B is required for parasympathetic enhancement of insulin secretion using an animal model and in vitro targeted knockdown/rescue experiments. We have also identified a mechanism by which ankyrin-B influences carbachol-dependent insulin release by demonstrating that ankyrin-B is required for normal levels of InsP₃R in islets and insulinoma cells. Finally, we have identified a human R1788W mutation of ankyrin-B that both associates with diabetes and causes a loss of function in pancreatic islets. Screening for ankyrin-B mutations allows for the personalization of disease treatment strategies. For example, our data suggest that strategies that blunt postprandial hyperglycemia (26, 27) or promote glp-1 signaling (18) would be beneficial.

The foregoing is illustrative of the present invention, and is not to be construed as limiting thereof. The invention is defined by the following claims, with equivalents of the claims to be included therein.

TABLE 1
CLINICAL CHARACTERISTICS OF GENNID
PROBANDS AND R/W HETEROZYGOTES

| | GENNID probands | | R/W heterozygotes | | |
|---|---|---|---|---|---|
| Category | (n = 1022) | | (n = 13) | | p value |
| Sex, % male | 39.9% | | 31% | | 0.58 |
| Age, years | 61.3 | (13.1) | 62.8 | (10.6) | 0.63 |
| BMI, kg/m² (SD) | 30.2 | (8.1) | 34.5 | (8.6) | 0.13 |
| Fasting glucose, | 174.1 | (72.6) | 179.9 | (60.1) | 0.76 |
| mg/dL (SD) | | | | | |
| Total cholesterol, | 189.8 | (40.6) | 184.9 | (37.1) | 0.59 |
| mg/dL (SD) | | | | | |
| HDL, mg/dL (SD) | 38.2 | (11.1) | 38.2 | (9.9) | 1.00 |
| LDL, mg/dL (SD) | 118.4 | (33.9) | 112.6 | (25.5) | 0.45 |
| Triglycerides, | 168.8 | (150.2) | 130.0 | (41.2) | 0.82 |
| mg/dL (SD) | | | | | |

| | | | |
|---|---|---|---|
| History of | 17.1% | 15% | 1.00 |
| heart disease | | | |
| History of | 41.8% | 38% | 0.17 |
| hypertension | | | |
| History of | 4.1% | 7% | 1.00 |
| kidney disease | | | |

TABLE 2
Table 2
ank2 SNPs

| | | REF SEQ | NUCLEOTIDE | NUCLEOTIDE | |
|---|---|---|---|---|---|
| | | (IF | (WILD TYPE | (POLY- | SEQUENCE SURROUNDING NUCLEOTIDE |
| SNP | ENSEMBL GENE | AVAILABLE) | ALLELE) | MORPHISM) | CHANGE |
| E1425G | ENSG00000145362 | | A | G | GTTGAAAGGGTATTTATTTACTCTTTCCTTTTCTTCTAAA |
| | | | | | TACTCTCTACTCTTCCTTCTCTCTTTTTTCCATCTTGCAT |
| | | | | | GGCATCTTGGGGCGGAAAGG[A/G]ATCAGAGTCAGAT |
| | | | | | CAAGAACAGGAGGAAGAGGTAATTTTATGACAGTGTCA |
| | | | | | CTTGTTATCGGCTGTGTCATTGCTGTAACCACTAATAA |
| | | | | | GAGCACATAGT |
| R1450W | ENSG00000145362 | | C | T | CTTTCTTTGAATGAATCAGTACTGTGGTTCCTCTCCTGT |
| | | | | | CATAGACAACCTTTGGCCATTCTGTTTTTGACCTTCTCC |
| | | | | | AGATCCACAGGATGAGCAGGAA[C/T]GGATCGAGGAAA |
| | | | | | GGCTGGCTTATATTGCTGATCACCTTGGCTTCAGCTGG |
| | | | | | ACAGGTAAAAAGAATGTGACCCAGGTTTTCAACAAAAC |
| | | | | | CTGACATAGATG |
| V1516D | ENSG00000145362 | | T | A | TATCAAAAATTTAGTAAGGCAGTTGAGTGAAAGAGATTT |
| | | | | | TTAAGAGTACCTCTCAGACATAATAAATGCTGTTTCTCT |
| | | | | | AATGTGTCAGATACCAACCTCG[T/A]TGAATGTCTCACC |
| | | | | | AAGATCAACCGAATGGATATTGTTCATCTCATGGAGAC |
| | | | | | CAACACAGAACCTCTCCAGGAGCGCATCAGTCATAGTT |
| | | | | | ATGCAGAAATT |
| T1552N | ENSG00000145362 | rs45608232 | A | C | CTCACCAAGATCAACCGAATGGATATTGTTCATCTCAT |
| | | | | | GGAGACCAACACAGAACCTCTCCAGGAGCGCATCAGT |
| | | | | | CATAGTTATGCAGAAATTGAACAGA[A/C]CATTACACTG |
| | | | | | GATCATAGTGAAGGTCAAACTGTGTGTGTGTATGTGTG |
| | | | | | TGTGTGTGTGTGTGTGTGTGTGTGTTGTGTCTGTGTGT |
| | | | | | GGTTAATTGAGGCA |
| L1622I | ENSG00000145363 | | C | A | CTGAGGGGGACAGCTCAGCAACAGCA[C/A]TCTTTCCC |
| | | | | | CAAANTCACAAGGAGC |
| T1626N | ENSG00000145362 | | C | A | ATCGTCTCAGAGGAAGACATTTCTGTTGGTTATTCCAC |
| | | | | | TTTTCAGGATGGCGTCCCCAAAACTGAGGGGGACAGC |
| | | | | | TCAGCAACAGCACTCTTTCCCCAAA[C/A]TCACAAGGA |
| | | | | | GCAAGTTCAACAGGATTTCTCAGGGAAAATGCAAGACC |
| | | | | | TGCCTGAAGAGTCATCTCTGGAATATCAGCAGGAATAT |
| | | | | | TTGTGAGTTTCCAAA |
| R1788W | ENSG00000145362 | | C | T | ACAAACTTCCTGTTTAAAATTTATCAATTCCATGGTACT |
| | | | | | GTCACACAAAAATAAGATACACAAATGAAATACATTTCA |
| | | | | | GGTTACTAGGAAAATCATTAGG[C/T]GGTATGTATCCTC |
| | | | | | TGAAGGCACAGAGAAAGAAGAGATTATGGTGCAGGGA |
| | | | | | ATGCCACAGGAACCTGTCAACATCGAGGAAGGGGATG |
| | | | | | GCTATTCCAAAGT |
| S1791P | ENSG00000145362 | | T | C | CTGTTTAAAATTTATCAATTCCATGGTACTGTCACACAA |
| | | | | | AAATAAGATACACAAATGAAATACATTTCAGGTTACTAG |
| | | | | | GAAAATCATTAGGCGGTATGTA[T/C]CCTCTGAAGGCA |
| | | | | | CAGAGAAAGAAGAGATTATGGTGCAGGGAATGCCACA |
| | | | | | GGAACCTGTCAACATCGAGGAAGGGGATGGCTATTCC |
| | | | | | AAAGTTATAAAGCG |
| E1813K | ENSG00000145363 | | G | A | ATTTCAGGTTACTAGGAAAATCATTAGGNGGTATGTAN |
| | | | | | CCTCTGAAGGCACAGAGAAAGAAGAGATTATGGTGCA |
| | | | | | GGGAATGCCACAGGAACCTGTCAACATC[G/A]AGGAAG |
| | | | | | GGGATGGCTATTCCAAAGTTATAAAGCGTGTTGTATTG |
| | | | | | AAGAGTGACACCGAGCAGT |
| V1516I | ENSG00000145362 | | G | A | ATATCAAAAATTTAGTAAGGCAGTTGAGTGAAAGAGAT |
| | | | | | TTTTAAGAGTACCTCTCAGACATAATAAATGCTGTTTCT |
| | | | | | CTAATGTGTCAGATACCAACCTC[G/A]TTGAATGTCTCA |
| | | | | | CCAAGATCAACCGAATGGATATTGTTCATCTCATGGAG |
| | | | | | ACCAACACAGAACCTCTCCAGGAGCGCATCAGTCATA |
| | | | | | GTTATGCAGAAAT |
| ANK2_ | ENSG00000145362 | | G | A | TTGAATGAATCAGTACTGTGGTTCCTCTCCTGTCATAG |
| E1452K | | | | | ACAACCTTTGGCCATTCTGTTTTTGACCTTCTCCAGATC |
| | | | | | CACAGGATGAGCAGGAACGGATC[G/A]AGGAAAGGCT |
| | | | | | GGCTTATATTGCTGATCACCTTGGCTTCAGCTGGACAG |
| | | | | | GTAAAAAGAATGTGACCCAGGTTTTTCAACAAAACCTGA |
| | | | | | CATAGATGCATCAG |
| ANK2_ | ENSG00000145362 | | G | C | TACCTCCAGACCCCAACATCCAGCGAGCGGGGAGGCT |
| S1721T | | | | | CTCCCATCATACAAGAACCCGAAGAGCCCTCAGAGCA |
| | | | | | CAGAGAGGAGAGCTCTCCGCGGAAAA[G/C]CAGCCTC |
| | | | | | GTAATAGTGGAGTCTGCCGATAACCAGCCTGAGACCT |
| | | | | | GTGAAAGACTCGATGAAGATGCAGCTTTTGAAAAGGTA |
| | | | | | AGACATTCCTCTCCACTT |
| ANK2_ | ENSG00000145362 | | C | A | TACCTCCAGACCCCAACATCCAGCGAGCGGGGAGGCT |
| T1726N | | | | | CTCCCATCATACAAGAACCCGAAGAGCCCTCAGAGCA |
| | | | | | CAGAGAGGAGAGCTCTCCGCGGAAAA[C/A]CAGCCTC |
| | | | | | GTAATAGTGGAGTCTGCCGATAACCAGCCTGAGACCT |
| | | | | | GTGAAAGACTCGATGAAGATGCAGCTTTTGAAAAGGTA |
| | | | | | AGACATTCCTCTCCACTT |
| ANK2_ | ENSG00000145362 | | G | A | CAATATAGGTAAGCTTCAACTAAATACTTAAATCATTCT |
| E1576K | | | | | GCCTTTAGGGTTCTCGGTACTTCAAGAGGAGTTATGCA |
| | | | | | CTGCACAGCACAAGCAGAAAGAG[G/A]AGCAAGCTGTT |
| | | | | | TCTAAAGAAAGTGAGACCTGCGATCACCCTCCTATCGT |
| | | | | | CTCAGAGGAAGACATTTCTGTTGGTTATTCCACTTTTCA |
| | | | | | GGATGGCGTCCC |
| | ENSG00000145362 | rs34270799 | A | C | CAGATAGGGGTGATGATTCTCCCGATTCTTCCCCAGAA |
| | | | | | GAACAGAAATCAGTAATCGAGATTCCTACTGCACCCAT |
| | | | | | GGAGAATGTGCCTTTTACTGAAAG[A/C]AAATCCAAAAT |
| | | | | | TCCTGTAAGGACTATGCCCACTTCCACCCCAGCACCTC |
| | | | | | CATCTGCAGAGTATGAGAGTTCAGTTTCTGAAGATTTT |
| | | | | | CTATCCAGTGTAG |

| | | SNP EXONAL LOCATION | | |
|---|---|---|---|---|
| | | IN PUBLISHED MRNA | AMINO ACID | AMINO ACID CHANGE |
| SNP | CHROMOSOMAL LOCATION | TRANSCRIPTS | (WILD TYPE ALLELE) | (SNP) |
| E1425G | chr4:114487882-114469882 | ENST00000264366, exon36; | E1425 | G |
| | | ENST06000343056, exon36; | | |
| | | ENST00000357077, exon36; | | |
| | | ENST00000361149, exon35; | | |
| | | ENST00000394537, exon36; | | |
| R1450W | chr4:114500448-114502448 | ENST00000264366, exon38; | R1450 | W |
| | | ENST00000343056, exon39; | | |
| | | ENST00000357077, exon39; | | |
| | | ENST00000361149, exon38; | | |
| | | ENST00000394537, exon39; | | |
| V1516D | chr4:114504656-114506656 | ENST00000264366, exon40; | V1516 | D |
| | | ENST00000343056, exon41; | | |
| | | ENST00000357077, exon41; | | |
| | | ENST00000361149, exon40; | | |
| | | ENST00000394537, exon41; | | |
| T1552N | chr4:114504764-114506764 | ENST00000264366, exon40; | T1552 | N |
| | | ENST00000343056, exon41; | | |
| | | ENST00000357077, exon41; | | |
| | | ENST00000361149, exon40; | | |
| | | ENST00000394537, exon41; | | |
| L1622I | chr4:114288904-114288964 | ENST00000264366, exon40; | L1622 | I |
| | | ENST00000343056, exon41; | | |
| | | ENST00000357077, exon41; | | |
| | | ENST00000361149, exon40; | | |
| | | ENST00000394537, exon41; | | |
| T1626N | chr4:114507369-114509369 | ENST00000264366, exon41; | T1626 | N |
| | | ENST00000343056, exon42; | | |
| | | ENST00000357077, exon42; | | |
| | | ENST00000361149, exon41; | | |
| | | ENST00000394537, exon42; | | |
| R1788W | chr4:114512911-114514911 | ENST00000264366, exon44; | R1788 | W |
| | | ENST00000343056, exon45; | | |
| | | ENST00000357077, exon45; | | |
| | | ENST00000361149, exon44; | | |
| | | ENST00000394537, exon45; | | |
| S1791P | chr4:114512920-114514920 | ENST00000264366, exon44; | S1791 | P |
| | | ENST00000343056, exon45; | | |
| | | ENST00000357077, exon45; | | |
| | | ENST00000361149, exon44; | | |
| | | ENST00000394537, exon45; | | |
| E1813K | chr4:114512920-114514920 | ENST00000264366, exon42; | E1813 | K |
| | | ENST00000343056, exon43; | | |
| | | ENST00000357077, exon43; | | |
| | | ENST00000361149, exon42; | | |
| | | ENST00000394537, exon43; | | |
| V1516I | chr4:114504655-114506655 | ENST00000264366, exon40; | V1516 | I |
| | | ENST00000343056, exon41; | | |
| | | ENST00000357077, exon41; | | |
| | | ENST00000361149, exon40; | | |
| | | ENST00000394537, exon41; | | |
| ANK2_ | chr4:114500454-114502454 | ENST00000264366, exon38; | E1452 | K |
| E1452K | | ENST00000343056, exon39; | | |
| | | ENST00000357077, exon39; | | |
| | | ENST00000361149, exon38; | | |
| | | ENST00000394537, exon39; | | |
| ANK2_ | chr4:114509316-114511316 | ENST00000264366, exon42; | S1721 | I |
| S1721T | | ENST00000343056, exon43; | | |
| | | ENST00000357077, exon43; | | |
| | | ENST00000361149, exon42; | | |
| | | ENST00000394537, exon43; | | |
| ANK2_ | chr4:114509331-114511331 | ENST00000264366, exon42; | T1726 | N |
| T1726N | | ENST00000343056, exon43; | | |
| | | ENST00000357077, exon43; | | |
| | | ENST00000361149, exon42; | | |
| | | ENST00000394537, exon43; | | |
| ANK2_ | chr4:114509224-114509224 | ENST00000264366, exon41, | E1578 | K |
| E1578K | | ENST00000343056, exon42; | | |
| | | ENST00000357077, exon42; | | |
| | | ENST00000361149, exon41; | | |
| | | ENST00000394537, exon42; | | |
| | chr4:114498123-114500123 | ENST00000264366, intron37; | | |
| | | ENST00000343056, intron37; | | |
| | | ENST00000357077, exon38; | | |
| | | ENST00000361149, exon37; | | |
| | | ENST00000394537, exon38; | | |

### REFERENCES

1. B. Ahren, Diabetologia 43, 393 (April, 2000).
2. J. Iversen, Diabetes 22, 381 (May, 1973).
3. S. R. Bloom, A. V. Edwards, J Physiol 362, 303 (May, 1985).
4. L. Benthem, T. O. Mundinger, G. J. Taborsky, Jr., Am J Physiol Endocrinol Metab 278, E603 (April, 2000).
5. P. J. Mohler, J. Q. Davis, V. Bennett, PLoS Biol 3, e423 (December, 2005).
6. P. J. Mohler et al., J Biol Chem 279, 12980 (Mar. 26, 2004).
7. P. J. Mohler et al., Nature 421, 634 (Feb. 6, 2003).
8. P. J. Mohler et al., Proc Natl Acad Sci USA 101, 9137 (Jun. 15, 2004).
9. P. J. Mohler et al., PLoS ONE 2, e1051 (2007).
10. T. Nilsson, P. Arkhammar, A. Hallberg, B. Hellman, P. O. Berggren, Biochem J 248, 329 (Dec. 1, 1987).
11. T. Nilsson, J. Zwiller, A. L. Boynton, P. O. Berggren, FEBS Lett 229, 211 (Feb. 29, 1988).
12. E. Gagerman, L. A. Idahl, H. P. Meissner, I. B. Taljedal, Am J Physiol 235, E493 (November, 1978).
13. M. C. Garcia, M. P. Hermans, J. C. Henquin, Biochem J 254, 211 (Aug. 15, 1988).
14. P. Gilon, J. C. Henquin, Endocr Rev 22, 565 (October, 2001).
15. P. O. Berggren et al., Cell 119, 273 (Oct. 15, 2004).
16. C. B. Wollheim, E. G. Siegel, G. W. Sharp, Endocrinology 107, 924 (October, 1980).
17. S. N. Yang, P. O. Berggren, Endocr Rev 27, 621 (October, 2006).
18. D. J. Drucker, Cell Metab 3, 153 (March, 2006).
19. E. Cerasi, R. Luft, Acta Endocrinol (Copenh) 55, 278 (June, 1967).
20. M. J. Davies et al., Diabet Med 11, 432 (June, 1994).
21. S. Del Prato, A. Tiengo, Diabetes Metab Res Rev 17, 164 (May-June, 2001).
22. L. J. Raffel et al., Diabetes Care 19, 864 (August, 1996).
23. P. J. Mohler et al., Circulation 115, 432 (Jan. 30, 2007).
24. S. R. Cunha, P. J. Mohler, J Biol Chem 283, 31968 (Nov. 14, 2008).
25. L. S. Satin, Endocrine 13, 251 (December, 2000).
26. J. L. Chiasson et al., Lancet 359, 2072 (Jun. 15, 2002).
27. F. A. Van de Laar, P. L. Lucassen, R. P. Akkermans, E. H. Van de Lisdonk, W. J. De Grauw, Cochrane Database Syst Rev, CD005061 (2006).
28. H. E. Hohmeier et al., Diabetes 49, 424 (March, 2000).
29. J. R. Bain, J. C. Schisler, K. Takeuchi, C. B. Newgard, T. C. Becker, Diabetes 53, 2190 (September, 2004).
30. N. Maeda, M. Niinobe, K. Mikoshiba, Embo J 9, 61 (January, 1990).

1. A method of identifying a subject as having an increased risk of developing type 2 diabetes, the method comprising detecting in the subject the presence or absence of an ankB loss of function allele, wherein the presence of the ankB loss of function allele identifies the subject as having an increased risk of developing type 2 diabetes.

2. The method of claim 1, further comprising:
correlating the presence or absence of an ankB loss of function allele with the risk of developing type 2 diabetes

3. The method of claim 1, wherein the presence of the ankB loss of function allele further identifies the subject as suitable for a treatment that reduces postprandial glycemic levels and/or suitable for treatment with an agent that enhances a glucagon-like peptide 1 (glp1) signaling pathway.

4. The method of claim 1, wherein the method further comprises placing the subject identified as at risk for developing type 2 diabetes on a treatment that reduces postprandial glycemic levels and/or administering to the subject an agent that enhances a glp-1 signaling pathway.

5-7. (canceled)

8. The method of claim 1, wherein the method further comprises administering a gastric inhibitory peptide (GIP) analog to the subject identified as at risk for developing type 2 diabetes.

9. A method of treating a subject with type 2 diabetes, the method comprising:
identifying a subject with type 2 diabetes and an ankB loss of function allele; and
administering an agent that enhances a glucagon-like peptide 1 (glp-1) signaling pathway to the subject, thereby treating a subject with type 2 diabetes.

10. The method of claim 9, wherein the method further comprises detecting the presence of the ankB loss of function allele in the subject with type 2 diabetes.

11. A method of correlating an ankB loss of function allele with the risk of developing type 2 diabetes in a subject, the method comprising:
detecting the presence of the ankB loss of function allele in a plurality of subjects with type 2 diabetes to determine the prevalence of the ankB loss of function allele in the plurality of diabetic subjects; and
correlating the prevalence of the ankB loss of function allele with development of type 2 diabetes, thereby correlating the ankB loss of function allele with the risk of developing type 2 diabetes in a subject.

12. The method of claim 11, wherein the method further comprises comparing the prevalence of the ankB loss of function allele in the plurality of subjects with type 2 diabetes with the prevalence of the ankB loss of function allele in a plurality of subjects that do not have type 2 diabetes.

13. The method of claim 11, wherein the method further comprises:
detecting the presence or absence of the ankB loss of function allele in a subject; and
determining the risk of the subject developing type 2 diabetes.

14. A method of correlating the presence of an ankB loss of function allele with an effective treatment for preventing the development of type 2 diabetes in a subject that has the ankB loss of function allele, the method comprising:
administering a treatment to the subject that has the ankB loss of function allele; and
correlating the presence of the ankB loss of function allele with the effectiveness of the treatment for preventing the development of type 2 diabetes in the subject.

15. The method of claim 14, further comprising:
determining the effectiveness of the treatment for treating type 2 diabetes in the subject.

16. A computer-assisted method of identifying an effective treatment for type 2 diabetes in a subject having an ankB loss of function allele that is associated with type 2 diabetes, the method comprising:
(a) storing a database of biological data for a plurality of subjects, the biological data that is being stored including for each of said plurality of subjects:
(i) a treatment type,
(ii) an ankB loss of function allele associated with type 2 diabetes, and
(iii) at least one clinical measure for type 2 diabetes from which treatment efficacy can be determined; and then
(b) querying the database to determine the effectiveness of a treatment type in treating type 2 diabetes in a subject having an ankB loss of function allele, thereby identifying an effective treatment for type 2 diabetes in a subject having an ankB loss of function allele associated with type 2 diabetes.

17. A method of correlating an ankB loss of function allele with a good or poor prognosis for type 2 diabetes, the method comprising:
detecting the presence or absence of the ankB loss of function allele in a plurality of subjects with type 2 diabetes; and
correlating the presence or absence of the ankB loss of function allele with a good or poor prognosis for type 2 diabetes in the plurality of subjects, thereby correlating the ankB loss of function allele with a good or poor prognosis for type 2 diabetes in a subject.

18. A method of identifying a subject with type 2 diabetes as having a good or a poor disease prognosis, the method comprising:
correlating the presence or absence of an ankB loss of function allele with a good or a poor prognosis for type 2 diabetes; and
determining the presence or absence of the ankB loss of function allele in a subject, wherein the presence or absence of the ankB loss of function allele identifies the subject as having a good or a poor disease prognosis.

19. The method of claim 1, wherein the subject is a human subject.

20. The method of claim 19, wherein the ankB loss of function allele comprises an amino acid substitution in the ankB amino acid sequence as compared with NCBI database Accession No. GI:119626696.

21. The method of claim 19, wherein the ankB loss of function allele results in:
(a) a glutamic acid to glycine substitution at amino acid position 1425 of ankyrin-B relative to NCBI database Accession No. GI:119626696 (FIG. 1; SEQ ID NO:1);
(b) an arginine to tryptophan substitution at amino acid position 1450 of ankyrin-B relative to NCBI database Accession No. GI:119626696;
(c) a valine to aspartic acid substitution at amino acid position 1516 of ankyrin-B relative to NCBI database Accession No. GI:119626696;
(d) a threonine to asparagine substitution at amino acid position 1552 of ankyrin-B relative to NCBI database Accession No. GI:119626696;
(e) a leucine to isoleucine substitution at amino acid position 1622 of ankyrin-B relative to NCBI database Accession No. GI:119626696;
(f) a threonine to asparagine substitution at amino acid position 1626 of ankyrin-B relative to NCBI database Accession No. GI:119626696;
(g) an arginine to tryptophan substitution at amino acid position 1788 of ankyrin-B relative to NCBI database Accession No. GI:119626696;
(h) a serine to proline substitution at amino acid position 1791 of ankyrin-B relative to NCBI database Accession No. GI:119626696;
(i) a glutamic acid to lysine substitution at amino acid position 1813 of ankyrin-B relative to NCBI database Accession No. GI:119626696;
(j) a valine to methionine substitution at amino acid position 1777 of ankyrin-B relative to NCBI database Accession No. GI:119626696;
(k) an arginine to isoleucine substitution at amino acid position 1404 of ankyrin-B relative to NCBI database Accession No. GI:119626696;
(l) a valine to isoleucine substitution at amino acid position 1516 of ankyrin-B relative to NCBI database Accession No. GI:119626696;
(m) a glutamic acid to lysine substitution at amino acid position 1452 of ankyrin-B relative to NCBI database Accession No. GI:119626696;
(n) a serine to threonine substitution at amino acid position 1721 of ankyrin-B relative to NCBI database Accession No. GI:119626696;
(o) a threonine to asparagine substitution at amino acid position 1726 of ankyrin-B relative to NCBI database Accession No. GI:119626696;
(p) a glutamic acid to lysine substitution at amino acid position 1578 of ankyrin-B relative to NCBI database Accession No. GI:119626696; or
(q) any combination of (a) to (p).

22. The method of claim 1, wherein the presence or absence of the ankB loss of function allele is determined from the amino acid sequence of ankyrin-B produced in the subject.

23. The method of claim 1, wherein the presence or absence of the ankB loss of function allele is determined from the nucleotide sequence of ankB in nucleic acid of the subject.
